(19) United States
(12) Patent Application Publication
(10) Pub. No.: US 2020/0085782 A1
(43) Pub. Date: Mar. 19, 2020

(54) COMPOSITIONS AND METHODS OF ENHANCING OR AUGMENTING TYPE I IFN PRODUCTION

(71) Applicant: Mavupharma, Inc., Kirkland, WA (US)

(72) Inventors: William Michael GALLATIN, Kirkland, WA (US); Gregory N. DIETSCH, Kirkland, WA (US)

(21) Appl. No.: 16/470,526

(22) PCT Filed: Dec. 21, 2017
(86) PCT No.: PCT/US2017/068041

§ 371 Date: Jun. 17, 2019

Related U.S. Application Data
(60) Provisional application No. 62/438,244, filed on Dec. 22, 2016.

(51) Int. Cl. A61K 31/353; A61K 31/52; A61K 31/517; A61K 45/06

(52) U.S. Cl. CPC A61K 31/353; A61K 45/06; A61K 31/517; A61K 31/52

(57) ABSTRACT

Disclosed herein are methods and compounds of augmenting and enhancing the production of type I IFNs in vivo. In some embodiments, also disclosed herein include methods of activating and enhancing the cGAS-STING response and use of an immunogenic cell death inducer with an inhibitor of a phosphodiesterase for the treatment of cancer.

### CROSS-REFERENCE

This patent application claims the benefit of U.S. Provisional Patent Application No. 62/438,244, filed Dec. 22, 2016, which is incorporated herein by reference in its entirety.

### BACKGROUND OF THE DISCLOSURE

Cancer immunotherapy comprises the use of the patient's immune system to combat tumor cells. In some instances, cancer immunotherapy utilizes the presence of tumor antigens (e.g., tumor-specific antigens) to facilitate the recognition of the tumor cells by the immune system. In other instances, cancer immunotherapy utilizes immune system components such as lymphocytes and cytokines to coordinate a general immune response.

### SUMMARY OF THE DISCLOSURE

Disclosed herein, in certain embodiments, are methods of augmenting and/or enhancing the production of type I IFNs in vivo. In some embodiments, the method localizes the production of type I IFNs within the tumor microenvironment. In some embodiments, also disclosed herein are methods of activating and enhancing the cGAS-STING response. In other embodiments, described herein comprise methods of priming a cancer with an immunogenic cell death inducer prior to stimulating the cGAS-STING pathway. In additional embodiments, described herein comprise use of an inhibitor of a 2′3′-cGAMP degradation polypeptide (e.g., an inhibitor of a phosphodiesterase) to block the 2′3′-cGAMP degradation polypeptide prior to priming a cancer with an immunogenic cell death inducer and use of an inhibitor of a 2′3′-cGAMP degradation polypeptide (e.g., an inhibitor of a phosphodiesterase) with an immunogenic cell death inducer for the treatment of a cancer. In some cases, further described herein are designs and generation of selective inhibitors to prevent the degradation of a STING activating substrate and pharmaceutical compositions comprising the selective inhibitor.

Disclosed herein, in certain embodiments, is a method of treating a subject having a cancer primed by an immunogenic cell death (ICD) inducer, comprising: administering to the subject a phosphodiesterase (PDE) inhibitor, wherein the PDE inhibitor prevents hydrolysis of 2′3′-cGAMP. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor is a small molecule. In some embodiments, the PDE inhibitor is a ENPP-1 inhibitor. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor binds to the catalytic domain of ENPP-1. In some embodiments, the PDE inhibitor binds to the nuclease-like domain of ENPP-1. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the cancer is a solid tumor. In some embodiments, the solid tumor comprises breast cancer, lung cancer or glioblastoma. In some embodiments, the cancer is a hematologic malignancy. In some embodiments, the hematologic malignancy is a leukemia, a lymphoma or a myeloma. In some embodiments, the hematologic malignancy is a B-cell malignancy. In some embodiments, the hematologic malignancy comprises multiple myeloma. In some embodiments, the cancer is a relapsed or refractory cancer. In some embodiments, the cancer is a metastatic cancer. In some embodiments, the immunogenic cell death (ICD) inducer comprises radiation. In some embodiments, the radiation comprises UV radiation. In some embodiments, the radiation comprises γ radiation. In some embodiments, the ICD inducer comprises a small molecule compound or a biologic. In some embodiments, the ICD inducer comprises a chemotherapeutic agent. In some embodiments, the chemotherapeutic agent comprises an antracycline. In some embodiments, the antracycline is doxorubicin or mitoxantrone. In some embodiments, the chemotherapeutic agent comprises a cyclophosphamide. In some embodiments, the cyclophosphamide is mafosfamide. In some embodiments, the chemotherapeutic agent is selected from bortezomib, daunorubicin, docetaxel, oxaliplatin, paclitaxel, or a combination thereof. In some embodiments, the ICD inducer comprises digitoxin or digoxin. In some embodiments, the ICD inducer comprises septacidin. In some embodiments, the ICD inducer comprises a combination of cisplatin and thapsigargin. In some embodiments, the ICD inducer comprises a combination of cisplatin and tunicamycin. In some embodiments, the ICD inducer comprises trastuzumab emtansine. In some embodiments, the ICD inducer comprises an activator of calreticulin (CRT) exposure. In some embodiments, the PDE inhibitor is administered to the subject at least 0.5, 1, 1.5, 2, 3, 4, 5, 6, 7, 8, 9, 10, 11, 12, 18, 24, 36 or 48 hours after administration of the ICD inducer. In some embodiments, the PDE inhibitor is administered to the subject at least 1, 2, 3, 4, 5, 6, 7, 8, 9, 10, 11, 12, 13, 14, 28, 30 or 40 days after administration of the ICD inducer. In some embodiments, the PDE inhibitor is administered continuously for 1, 2, 3, 4, 5, 6, 7, 8, 9, 10, 14, 15, 28, 30 or more days. In some embodiments, the PDE inhibitor is administered at predetermined time intervals for 1, 2, 3, 4, 5, 6, 7, 8, 9, 10, 14, 15, 28, 30 or more days. In some embodiments, the PDE inhibitor is administered intermittently for 1, 2, 3, 4, 5, 6, 7, 8, 9, 10, 14, 15, 28, 30 or more days. In some embodiments, the PDE inhibitor and the ICD inducer are administered for at least 1 cycle, 2 cycles, 3 cycles, 4 cycles, 5 cycles or more. In some embodiments, each cycle comprises 14 to 28 days. In some embodiments, the PDE inhibitor is administered to the subject at a therapeutically effective amount. In some embodiments, the therapeutically effective amount is administered in 1 dose, 2 doses, 3 doses, 4 doses, 5 doses, 6 doses or more. In some embodiments, the therapeutically effective amount of the PDE inhibitor selectively inhibits hydrolysis of 2′3′-cGAMP. In some embodiments, the therapeutically effective amount of the PDE inhibitor reduces ATP hydrolysis in PDE by less than 50%, less than 40%, less than 30%, less than 20%, or by less than 10% relative to the ATP hydrolysis of a PDE in the absence of the PDE inhibitor. In some embodiments, the therapeutically effective amount of the PDE inhibitor does not induce ATP hydrolysis in PDE. In some embodiments, the method further comprises administering an additional therapeutic agent. In some embodiments, the additional therapeutic agent comprises an immune checkpoint inhibitor. In some embodiments, the PDE inhibitor and the additional therapeutic agent is administered simultaneously. In some embodiments, the PDE inhibitor and the additional therapeutic agent is administered sequentially. In some embodiments, the PDE inhibitor is administered before administering the additional therapeutic agent. In some embodiments, the PDE inhibitor is administered after administering the additional therapeutic agent. In some embodiments, the subject is a human. In some embodiments, the subject is diagnosed with the cancer. In some embodiments, the subject has a resistance to an immune checkpoint inhibitor prior to the administration of the inhibitor of PDE.

Disclosed herein, in certain embodiments, is a method of treating a subject having a cancer, comprising: administering to the subject a phosphodiesterase (PDE) inhibitor and an immunogenic cell death (ICD) inducer; wherein the PDE inhibitor prevents hydrolysis of 2′3′-cGAMP, and wherein the PDE inhibitor is administered either prior to administering the ICD inducer or simultaneously with the ICD inducer. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor is a small molecule. In some embodiments, the PDE inhibitor is a ENPP-1 inhibitor. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor binds to the catalytic domain of ENPP-1. In some embodiments, the PDE inhibitor binds to the nuclease-like domain of ENPP-1. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the cancer is a solid tumor. In some embodiments, the solid tumor comprises breast cancer, lung cancer or glioblastoma. In some embodiments, the cancer is a hematologic malignancy. In some embodiments, the hematologic malignancy is a leukemia, a lymphoma or a myeloma. In some embodiments, the hematologic malignancy is a B-cell malignancy. In some embodiments, the hematologic malignancy comprises multiple myeloma. In some embodiments, the cancer is a relapsed or refractory cancer. In some embodiments, the cancer is a metastatic cancer. In some embodiments, the immunogenic cell death (ICD) inducer comprises radiation. In some embodiments, the radiation comprises UV radiation. In some embodiments, the radiation comprises γ radiation. In some embodiments, the ICD inducer comprises a small molecule compound or a biologic. In some embodiments, the ICD inducer comprises a chemotherapeutic agent. In some embodiments, the chemotherapeutic agent comprises an antracycline. In some embodiments, the antracycline is doxorubicin or mitoxantrone. In some embodiments, the chemotherapeutic agent comprises a cyclophosphamide. In some embodiments, the cyclophosphamide is mafosfamide. In some embodiments, the chemotherapeutic agent is selected from bortezomib, daunorubicin, docetaxel, oxaliplatin, paclitaxel, or a combination thereof. In some embodiments, the ICD inducer comprises digitoxin or digoxin. In some embodiments, the ICD inducer comprises septacidin. In some embodiments, the ICD inducer comprises a combination of cisplatin and thapsigargin. In some embodiments, the ICD inducer comprises a combination of cisplatin and tunicamycin. In some embodiments, the ICD inducer comprises trastuzumab emtansine. In some embodiments, the ICD inducer comprises an activator of calreticulin (CRT) exposure. In some embodiments, the PDE inhibitor is administered to the subject at least 0.5, 1, 1.5, 2, 3, 4, 5, 6, 7, 8, 9, 10, 11, 12, 18, 24, 36 or 48 hours prior to administration of the ICD inducer. In some embodiments, the PDE inhibitor is administered to the subject at least 1, 2, 3, 4, 5, 6, 7, 8, 9, 10, 14, 15, 28, or 30 days prior to administration of the ICD inducer. In some embodiments, the PDE inhibitor is administered simultaneously with the ICD inducer. In some embodiments, the PDE inhibitor is administered continuously for 1, 2, 3, 4, 5, 6, 7, 8, 9, 10, 14, 15, 28, 30 or more days. In some embodiments, the PDE inhibitor is administered at predetermined time intervals for 1, 2, 3, 4, 5, 6, 7, 8, 9, 10, 14, 15, 28, 30 or more days. In some embodiments, the PDE inhibitor is administered intermittently for 1, 2, 3, 4, 5, 6, 7, 8, 9, 10, 14, 15, 28, 30 or more days. In some embodiments, the PDE inhibitor is administered simultaneously or sequentially with the ICD inducer for at least 1 cycle, 2 cycles, 3 cycles, 4 cycles, 5 cycles or more. In some embodiments, each cycle comprises 14 to 28 days. In some embodiments, the PDE inhibitor is administered to the subject at a therapeutically effective amount. In some embodiments, the therapeutically effective amount is administered in 1 dose, 2 doses, 3 doses, 4 doses, 5 doses, 6 doses or more. In some embodiments, the therapeutically effective amount of the PDE inhibitor selectively inhibits hydrolysis of 2′3′-cGAMP. In some embodiments, the therapeutically effective amount of the PDE inhibitor reduces ATP hydrolysis in PDE by less than 50%, less than 40%, less than 30%, less than 20%, less than 10%, less than 5% or by less than 1% relative to the ATP hydrolysis of a PDE in the absence of the PDE inhibitor. In some embodiments, the therapeutically effective amount of the PDE inhibitor does not induce ATP hydrolysis in PDE. In some embodiments, the method further comprises administering an additional therapeutic agent. In some embodiments, the additional therapeutic agent comprises an immune checkpoint inhibitor. In some embodiments, the PDE inhibitor and the additional therapeutic agent is administered simultaneously. In some embodiments, the PDE inhibitor and the additional therapeutic agent is administered sequentially. In some embodiments, the PDE inhibitor is administered before administering the additional therapeutic agent. In some embodiments, the PDE inhibitor is administered after administering the additional therapeutic agent. In some embodiments, the subject is a human. In some embodiments, the subject is diagnosed with the cancer. In some embodiments, the subject has a resistance to an immune checkpoint inhibitor prior to the administration of the inhibitor of PDE.

Disclosed herein, in certain embodiments, is a method of inhibiting depletion of 2′3′-cGAMP in a cell, comprising: contacting a cell comprising a 2′3′-cGAMP degradation polypeptide with an inhibitor to generate a 2′3′-cGAMP degradation polypeptide-inhibitor adduct, thereby inhibiting the 2′3′-cGAMP degradation polypeptide from degrading 2′3′-cGAMP to prevent the depletion of 2′3′-cGAMP in the cell. In some embodiments, the 2′3′-cGAMP degradation polypeptide is a phosphodiesterase (PDE). In some embodiments, the 2′3′-cGAMP degradation polypeptide is an ectonucleotide pyrophosphatase/phosphodiesterase (ENPP) protein. In some embodiments, the 2′3′-cGAMP degradation polypeptide is ectonucleotide pyrophosphatase/phosphodiesterase family member 1 (ENPP-1). In some embodiments, the cell has an elevated expression of PDE. In some embodiments, the cell has an elevated population of cytosolic DNA. In some embodiments, the elevated population of cytosolic DNA is generated by an ICD-mediated event. In some embodiments, the elevated population of cytosolic DNA is generated by DNA structure-specific endonuclease MUS81. In some embodiments, the inhibitor is a PDE inhibitor. In some embodiments, the PDE inhibitor is a small molecule. In some embodiments, the PDE inhibitor is an ENPP-1 inhibitor. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor binds to the catalytic domain of ENPP-1. In some embodiments, the PDE inhibitor binds to the nuclease-like domain of ENPP-1. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the cell is a tumor cell. In some embodiments, the tumor cell is a solid tumor cell. In some embodiments, the tumor cell is a blood cancer cell. In some embodiments, the cell is an effector cell. In some embodiments, the effector cell is a dendritic cell or a macrophage. In some embodiments, the cell is further contacted with a recombinant vaccine. In some embodiments, the recombinant vaccine comprises a vector encoding a tumor antigen. In some embodiments, the vector is a plasmid vector or a viral vector, optionally a vector selected from an adenoviral based vector, an adeno-associated viral based vector, or a lentiviral based vector. In some embodiments, the method is an in vivo method.

Disclosed herein, in certain embodiments, is a method of enhancing type I interferon (IFN) production in a subject in need thereof, comprising: administering to the subject a pharmaceutical composition comprising: (i) an inhibitor of a 2′3′-cGAMP degradation polypeptide to block the hydrolysis of 2′3′-cGAMP; and (ii) a pharmaceutically acceptable excipient; wherein the presence of 2′3′-cGAMP activates the STING pathway, thereby enhancing the production of type I interferons. In some embodiments, the production of IFNs is localized in a tumor microenvironment. In some embodiments, the 2′3′-cGAMP degradation polypeptide is a phosphodiesterase (PDE). In some embodiments, the 2′3′-cGAMP degradation polypeptide is an ectonucleotide pyrophosphatase/phosphodiesterase (ENPP) protein. In some embodiments, the 2′3′-cGAMP degradation polypeptide is ectonucleotide pyrophosphatase/phosphodiesterase family member 1 (ENPP-1). In some embodiments, the cell has an elevated expression of PDE. In some embodiments, the cell has an elevated population of cytosolic DNA. In some embodiments, the elevated population of cytosolic DNA is generated by an ICD-mediated event. In some embodiments, the elevated population of cytosolic DNA is generated by DNA structure-specific endonuclease MUS81. In some embodiments, the inhibitor is a PDE inhibitor. In some embodiments, the PDE inhibitor is a small molecule. In some embodiments, the PDE inhibitor is an ENPP-1 inhibitor. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor binds to the catalytic domain of ENPP-1. In some embodiments, the PDE inhibitor binds to the nuclease-like domain of ENPP-1. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the subject has been administered an immunogenic cell death (ICD) inducer prior to administering the inhibitor of a 2′3′-cGAMP degradation polypeptide. In some embodiments, the immunogenic cell death (ICD) inducer comprises radiation. In some embodiments, the radiation comprises UV radiation. In some embodiments, the radiation comprises γ radiation. In some embodiments, the ICD inducer comprises a small molecule compound or a biologic. In some embodiments, the ICD inducer comprises a chemotherapeutic agent. In some embodiments, the chemotherapeutic agent comprises an antracycline. In some embodiments, the antracycline is doxorubicin or mitoxantrone. In some embodiments, the chemotherapeutic agent comprises a cyclophosphamide. In some embodiments, the cyclophosphamide is mafosfamide. In some embodiments, the chemotherapeutic agent is selected from bortezomib, daunorubicin, docetaxel, oxaliplatin, paclitaxel, or a combination thereof. In some embodiments, the ICD inducer comprises digitoxin or digoxin. In some embodiments, the ICD inducer comprises septacidin. In some embodiments, the ICD inducer comprises a combination of cisplatin and thapsigargin. In some embodiments, the ICD inducer comprises a combination of cisplatin and tunicamycin. In some embodiments, the ICD inducer comprises trastuzumab emtansine. In some embodiments, the ICD inducer comprises an activator of calreticulin (CRT) exposure. In some embodiments, the inhibitor of a 2′3′-cGAMP degradation polypeptide is administered to the subject at least 0.5, 1, 1.5, 2, 3, 4, 5, 6, 7, 8, 9, 10, 11, 12, 18, 24, 36 or 48 hours after administration of the ICD inducer. In some embodiments, the inhibitor of a 2′3′-cGAMP degradation polypeptide is administered to the subject at least 1, 2, 3, 4, 5, 6, 7, 8, 9, 10, 11, 12, 13, 14, 28, 30 or 40 days after administration of the ICD inducer. In some embodiments, the inhibitor of a 2′3′-cGAMP degradation polypeptide is administered to the subject at least 0.5, 1, 1.5, 2, 3, 4, 5, 6, 7, 8, 9, 10, 11, 12, 18, 24, 36 or 48 hours prior to administration of the ICD inducer. In some embodiments, the inhibitor of a 2′3′-cGAMP degradation polypeptide is administered to the subject at least 1, 2, 3, 4, 5, 6, 7, 8, 9, 10, 11, 12, 13, 14, 28, 30 or 40 days prior to administration of the ICD inducer. In some embodiments, the PDE inhibitor is administered simultaneously with the ICD inducer. In some embodiments, the inhibitor of a 2′3′-cGAMP degradation polypeptide is administered to the subject. In some embodiments, the inhibitor of a 2′3′-cGAMP degradation polypeptide is administered continuously for 1, 2, 3, 4, 5, 6, 7, 8, 9, 10, 14, 15, 28, 30 or more days. In some embodiments, the inhibitor of a 2′3′-cGAMP degradation polypeptide is administered at predetermined time intervals for 1, 2, 3, 4, 5, 6, 7, 8, 9, 10, 14, 15, 28, 30 or more days. In some embodiments, the inhibitor of a 2′3′-cGAMP degradation polypeptide is administered intermittently for 1, 2, 3, 4, 5, 6, 7, 8, 9, 10, 14, 15, 28, 30 or more days. In some embodiments, the inhibitor of a 2′3′-cGAMP degradation polypeptide is administered simultaneously or sequentially with the ICD inducer for at least 1 cycle, 2 cycles, 3 cycles, 4 cycles, 5 cycles or more. In some embodiments, each cycle comprises 14 to 28 days. In some embodiments, the inhibitor of a 2′3′-cGAMP degradation polypeptide is administered to the subject at a therapeutically effective amount. In some embodiments, the therapeutically effective amount is administered in 1 dose, 2 doses, 3 doses, 4 doses, 5 doses, 6 doses or more. In some embodiments, the therapeutically effective amount of the inhibitor of a 2′3′-cGAMP degradation polypeptide selectively inhibits hydrolysis of 2′3′-cGAMP but not ATP hydrolysis in the 2′3′-cGAMP degradation polypeptide. In some embodiments, the method further comprises administering an additional therapeutic agent. In some embodiments, the additional therapeutic agent comprises an immune checkpoint inhibitor. In some embodiments, the PDE inhibitor and the additional therapeutic agent is administered simultaneously. In some embodiments, the PDE inhibitor and the additional therapeutic agent is administered sequentially. In some embodiments, the PDE inhibitor is administered before the administration of the additional therapeutic agent. In some embodiments, the PDE inhibitor is administered after the administration of the additional therapeutic agent. In some embodiments, the subject is diagnosed with cancer. In some embodiments, the cancer is a solid tumor. In some embodiments, the solid tumor comprises breast cancer, lung cancer or glioblastoma. In some embodiments, the cancer is a hematologic malignancy. In some embodiments, the hematologic malignancy is a leukemia, a lymphoma or a myeloma. In some embodiments, the hematologic malignancy is a B-cell malignancy. In some embodiments, the hematologic malignancy comprises multiple myeloma. In some embodiments, the cancer is a relapsed or refractory cancer. In some embodiments, the cancer is a metastatic cancer. In some embodiments, the subject has a resistance to an immune checkpoint inhibitor prior to the administration of the inhibitor of a 2′3′-cGAMP degradation polypeptide.

Disclosed herein, in certain embodiments, is a method of stabilizing a stimulator of interferon genes (STING) protein dimer in a cell, comprising: (a) contacting a cell characterized with an elevated expression of a phosphodiesterase (PDE) or an elevated population of cytosolic DNA with a PDE inhibitor to inhibit hydrolysis of 2′3′-cGAMP; and (b) interacting 2′3′-cGAMP to a STING protein dimer to generate a 2′3′-cGAMP-STING complex, thereby stabilizing the STING protein dimer. In some embodiments, interacting 2′3′-cGAMP to a STING protein dimer to generate a 2′3′-cGAMP-STING complex further activates the STING protein dimer. In some embodiments, the method further comprises upregulating the production of type I interferon (IFN). In some embodiments, the production of IFNs is localized in a tumor microenvironment. In some embodiments, the elevated population of cytosolic DNA is generated by an ICD-mediated event. In some embodiments, the elevated population of cytosolic DNA is generated by DNA structure-specific endonuclease MUS81. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor is a small molecule. In some embodiments, the PDE inhibitor is an ENPP-1 inhibitor. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor binds to the catalytic domain of ENPP1. In some embodiments, the PDE inhibitor binds to the nuclease-like domain of ENPP1. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the cell is a tumor cell. In some embodiments, the tumor cell is a solid tumor cell. In some embodiments, the tumor cell is a blood cancer cell. In some embodiments, the cell is an effector cell. In some embodiments, the effector cell is a dendritic cell or a macrophage. In some embodiments, the method is an in vivo method.

Disclosed herein, in certain embodiments, is a method of selectively inhibits a phosphodiesterase (PDE), comprising: contacting a cell characterized with an elevated population of cytosolic DNA with a PDE inhibitor to inhibit hydrolysis of 2′3-cGAMP, wherein the PDE inhibitor has a reduced inhibition function of ATP hydrolysis of the PDE. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor binds to the catalytic domain of ENPP1. In some embodiments, the PDE inhibitor binds to the nuclease-like domain of ENPP1. In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the elevated population of cytosolic DNA is generated by an ICD-mediated event. In some embodiments, the elevated population of cytosolic DNA is generated by DNA structure-specific endonuclease MUS81. In some embodiments, the reduced inhibition function of ATP hydrolysis is relative to the ATP hydrolysis of a PDE in the absence of the PDE inhibitor. In some embodiments, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some embodiments, the PDE inhibitor does not inhibit ATP hydrolysis of the PDE. In some embodiments, the cell is a tumor cell. In some embodiments, the tumor cell is a solid tumor cell. In some embodiments, the tumor cell is a blood cancer cell. In some embodiments, the cell is an effector cell. In some embodiments, the effector cell is a dendritic cell or a macrophage. In some embodiments, the method is an in vivo method.

Disclosed herein, in certain embodiments, is a method of selectively inhibits a phosphodiesterase (PDE), comprising: contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the cell is a tumor cell. In some embodiments, the tumor cell is a solid tumor cell. In some embodiments, the tumor cell is a blood cancer cell. In some embodiments, the cell is an effector cell. In some embodiments, the effector cell is a dendritic cell or a macrophage. In some embodiments, the method is an in vivo method.

Disclosed herein, in certain embodiments, is a method of selectively inhibits a phosphodiesterase (PDE), comprising: contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the PDE inhibitor is a reversible inhibitor. In some embodiments, the PDE inhibitor is a competitive inhibitor. In some embodiments, the PDE inhibitor is an allosteric inhibitor. In some embodiments, the PDE inhibitor is an irreversible inhibitor. In some embodiments, the PDE inhibitor is a mixed inhibitor. In some embodiments, the cell is a tumor cell. In some embodiments, the tumor cell is a solid tumor cell. In some embodiments, the tumor cell is a blood cancer cell. In some embodiments, the cell is an effector cell. In some embodiments, the effector cell is a dendritic cell or a macrophage. In some embodiments, the method is an in vivo method.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a cartoon representation of the cGAS-STING pathway.

FIG. 2 illustrates a cartoon representation of an example of immunogenic tumor cell death mediating the induction of type I IFNs. After tumor implantation or radiation therapy, tumor-derived DNA can access the DC cytosol and bind cGAS to activate STING-mediated IFN transcription. 2′3′-cGAMP is generated by cGAS from the substrates ATP and GTP and, in turn, binds to and activates STING dimers, inducing phosphorylation of TBK-1 and IRF3. Nuclear translocation of phosphorylated IRF3 controls IFN-β transcription. After binding to its receptor, IFN-β renders DCs competent to present tumor antigens and prime CD8+ T lymphocytes. FIG. 2 is reproduced from Bronte, V. “Tumors STING adaptive antitumor immunity,” Immunity, 41: 679-681 (2014).

FIG. 3A-FIG. 3C are exemplary bar graphs illustrating augmentation of cGAMP mediated IFNβ production in the presence of PDE inhibitor Compound 1 (FIG. 3A), Compound 2 (FIG. 3B), and Compound 3 (FIG. 3C).

### DETAILED DESCRIPTION OF THE DISCLOSURE

In some embodiments, the immunophenotype of a tumor microenvironment modulates the responsiveness of the tumor to a cancer therapy. In some instances, tumor-infiltrating lymphocytes are correlated with favorable prognosis in different types of tumors and are correlated with positive clinical outcome in response to several lines of immunotherapy (Galon, et al., “Cancer classification using the immunoscore: a worldwide task force,” J. Transl. Med. 10:205, (2012); Postow, et al., “Targeting immune checkpoints: releasing the restraints on anti-tumor immunity for patients with melanoma,” Cancer J. 18: 153-159 (2012); Wolchok, et al., “Nivolumab plus ipilimumab in advanced melanoma,” N. Engl. J. Med. 369: 122-133 (2013)).

In some cases, innate immune sensing in the tumor microenvironment promotes T-cell priming and subsequent infiltration of tumor-infiltrating lymphocytes. For example, transcriptional profiling analyses of melanoma patients have shown that tumors containing infiltrating activated T cells are characterize by a type I IFN transcriptional signature (Harlin et al., “Chemokine expression in melanoma metastases associated with CD8+ T-cell recruitment,” Cancer Res. 69: 3077-3085 (2009). Furthermore, mice lacking the IFN-α/β receptor in dendritic cells are unable to reject immunogenic tumors and the CD8α+ dendritic cells from these mice are defective in antigen cross-presentation to CD8+ T cells (Fuertes, et al., “Host type I IFN signals are required for antitumor CD8+ T cell response through CD8alpha+ dendritic cells,” J. Exp. Med., 208: 2005-2015 (2011)).

In some embodiments, systemic delivery of type I IFNs has shown efficacy in cancer settings. Indeed, systemic injection of IFN-β in a mouse xenograft model of human colorectal cancer liver metastases has shown tumor regression and improved survival (Tada, et al., “Systemic IFN-β gene therapy results in long-term survival in mice with established colorectal liver metastases,” J. Clin. Invest. 108(1): 83-95 (2001)).

In some instances, systemic delivery of type I IFNs requires high doses to achieve therapeutic benefit. In such cases, desensitization of the immune system and issues with tolerability have also been observed.

In some embodiments, disclosed herein are methods of enhancing and/or augmenting the production of type I IFNs in vivo, without the need of systemic delivery of type I IFNs. In such instances, the IFN production is localized in the tumor microenvironment. In some cases, the methods comprise activating and enhancing the cGAS-STING response. In some cases, the methods comprise priming a cancer with an immunogenic cell death inducer prior to stimulating the cGAS-STING pathway. In other cases, the methods comprise blocking the degradation of a STING activating substrate prior to priming a cancer with an immunogenic cell death inducer. In additional cases, the methods comprise use of an inhibitor of a 2′3′-cGAMP degradation polypeptide (e.g., an inhibitor of a phosphodiesterase) with an immunogenic cell death inducer for the treatment of a cancer.

In additional embodiments, disclosed herein include methods of designing inhibitors of 2′3′-cGAMP degradation polypeptides and assays for evaluating the enzyme activity of the GMP degradation polypeptides.

cGAS-STING Pathway, Immunogenic Cell Death, and the Production of Type I IFNs

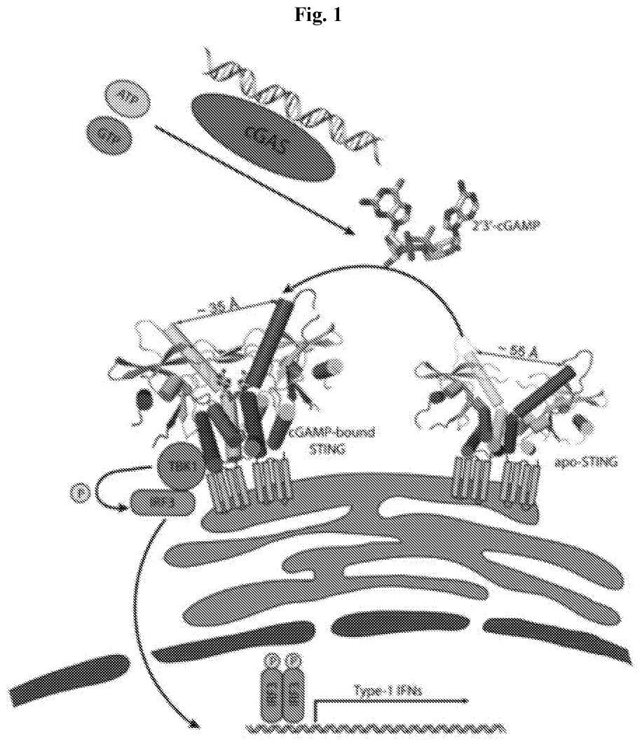

Cytosolic DNA can signal the presence of cellular damage and/or the presence of cancerous cells. These cytosolic DNAs (e.g., double stranded DNAs) are surveyed by DNA sensors such as RNA pol III, DAI, IFI116, DDX41, LSm14A, cyclic-GMP-AMP synthase, LRRFIP1, Sox2, DHX9/36, Ku70 and AIM2. Cyclic-GMP-AMP synthase (cGAS or cGAMP synthase) is a 522 amino acid protein that belongs to the nucleotidyltransferase family of cytosolic DNA sensors. Upon cytosolic DNA stimulation, cGAS synthesizes cGAMP, which comprises a first bond between the 2′-OH of GMP and the 5′-phosphate of AMP and a second bond between the 3′-OH of AMP and the 5′-phosphate of GMP. cGAMP (also known as cyclic GMP-AMP, 2′3′-cGAMP, cGAMP (2′-5′) or cyclic Gp(2′-5′)Ap(3′-5′)) serves as a ligand to STING, thereby activating the STING-mediated IFN (e.g., IFNβ) production (FIG. 1).

Mitochondria play a role in host immune response, for example, by boosting immune cell activation and antimicrobial defense. Mitochondrial DNA (mtDNA) triggers innate immune responses when exposed during cellular stress, infection or injury. Both cytosolic and extracellular mtDNA are recognized by DNA sensors and trigger type I interferons and interferon-stimulated gene (ISG) expression. In some instances, cytosolic mtDNA is recognized by DNA sensors and triggers type I interferons and interferon-stimulated gene (ISG) expression. In some instances, mtDNA is released during apoptosis mediated by BCL-2 like protein 4 (BAX) and BCL-2 homologous antagonist/killer (BAK). In some instances, mtDNA released during apoptosis engage cGAS-STING-IRF3 signaling and trigger type I IFN responses and expression of ISGs. In some instances, mitochondrial stress liberates cytosolic mtDNA which triggers type I IFN via the cGAS-STING pathway. In some instances, the stress is disease-mediated. In some instances, the disease is cancer. In some instances, extracellular mtDNA is recognized by DNA sensors and triggers type I interferons and interferon-stimulated gene (ISG) expression. Neutrophil extracellular trap (NET) formation—a process involved in bacterial clearance and sterile inflammatory diseases—results in cell death and extrusion of neutrophil DNA and/or protein complexes into the extracellular space. In some instances, extracellular mtDNA, such as mtDNA released from activated neutrophils, engage cGAS-STING pathway to trigger a type I IFN response.

In healthy cells cGAS is prevented from being activated by restricting the DNA to the nucleus and the mitochondria. The integrity of the nuclear envelope, in some cases, is critical for nuclear compartmentalization and for regulating the exchange of molecules between the nucleus and the cytoplasm. The nuclear envelope completely disassembles during cell division, and reassembles as the cell segregates the replicated DNA into daughter cells. In some instances, whole or broken chromosome fragments miss-segregate from the main chromatin mass. In some instances, the miss-segregated whole or broken chromosome fragments recruit nuclear envelope components to form micronuclei. In some instances, the micronuclei are compartmentally separated from the primary nucleus. In some instances, formation of micronuclei is induced by genome-instability. In some instances, formation of micronuclei is induced by cellular stress. In some instances, the nuclear envelope of the micronuclei disassembles. In some instances, the nuclear envelope of the micronuclei disassembles irreversibly. In some instances, the miss-segregated whole or broken chromosome fragments are not compartmentalized in the micronuclei due to the disassembled nuclear envelope. In some instances, the loss of compartmentalization of the miss-segregated whole or broken chromosome fragments in the micronuclei engage cGAS-STING pathway to trigger a type I IFN response.

In some instances, ligand for the cytosolic DNA sensor is nuclear DNA. In some instances, ligand for the cytosolic DNA sensor is mitochondrial DNA. In some instances, ligand for the cytosolic DNA sensor is cytosolic mitochondrial DNA. In some instances, ligand for the cytosolic DNA sensor is extracellular mitochondrial DNA. In some instances, ligand for the cytosolic DNA sensor localizes to a micronuclei. In some instances, ligand for the cytosolic DNA sensor is micronuclei with disassembled nuclear envelope.

STING (also known as stimulator of interferon genes, TMEM173, MITA, ERIS, or MPYS) is a 378 amino acid protein that comprises a N-terminal region containing four trans-membrane domains and a C-terminal domain that comprises a dimerization domain. Upon binding to 2′3′-cGAMP, STING undergoes a conformational rearrangement enclosing the 2′3′-cGAMP molecule.

Binding of 2′3′-cGAMP activates a cascade of events whereby STING recruits and activates IκB kinase (IKK) and TANK-binding kinase (TBK1), which following their phosphorylation, respectively activate nuclear transcription factor κB (NF-κB) and interferon regulatory factor 3 (IRF3). In some instances, the activated proteins translocate to the nucleus to induce transcription of the genes encoding type I IFN and cytokines for promoting intercellular host immune defense. In some cases, the production of type I IFNs further drives the development of cytolytic T cell response and enhances expression of MHC, thereby increasing antigen processing and presentation within a tumor microenvironment. In such cases, enhanced type I IFN production further renders the tumor cells to be more vulnerable by enhancing their recognition by the immune system.

In some instances, STING is capable of directly sensing bacterial cyclic dinucleotides (CDNs) such as c[di-GMP]. In some cases, 2′3′-cGAMP acts as a second messenger binding to STING in response to cells exposed to DNA.

In some embodiments, cytosolic DNA is generated through “self-DNA” or endogenous DNA from the host through the DNA structure-specific endonuclease methyl methane-sulphonate (MMS) and ultraviolet-sensitive 81 (MUS81) (Ho, et al., “The DNA structure-specific endonuclease MUS81 mediates DNA sensor STING-dependent host rejection of prostate cancer cells,” Immunity, 44:1177-1189 (2016)). The DNA structure-specific endonuclease MUS81 is a member of the XPF family of endonucleases that forms a heterodimeric complex with essential meiotic endonuclease 1 (EME1). In some instances, the MUS81-EME1 complex cleaves DNA structures at stalled replication forks. In some cases, MUS81 cleavage of self-DNA leads to accumulation of cytosolic DNA and activation of the STING pathway.

In other instances, cytosolic DNA is generated through immunogenic cell death (ICD)-mediated events, activation of the STING-pathway, production of type I INFs, and further priming of the tumor cell microenvironment.

### Immunogenic Cell Death

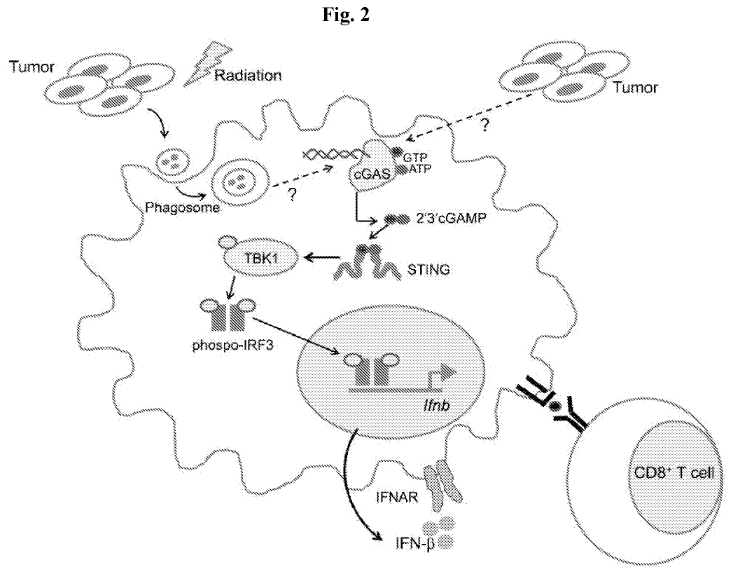

In some embodiments, immunogenic cell death (ICD), or immunogenic cancer cell death, is a cell death modality which further stimulates an immune response against tumor expressed antigens. In some cases, tumor expressed antigens are tumor neoantigens or antigens that are formed by mutated proteins and unique to the tumor. In other cases, tumor expressed antigens comprise overexpressed proteins such as MUC1, CA-125, MART-1 or carcinoembyonic antigen (CEA). In some instances, ICD is characterized by a series of biochemical events that comprises: 1) the cell surface translocation of calreticulin (CALR or CRT), an endoplasmic reticulum (ER) resident chaperone protein and a potent DC “eat me” signal; 2) the extracellular release of high mobility group box 1 (HMGB 1), a DNA binding protein and toll-like receptor 4 (TLR-4) mediated DC activator; and 3) the liberation of adenosine-5′-triphosphate (ATP), a cell-cell signaling factor in the extracellular matrix (ECM) that serves to activate P2X7 purinergic receptors on DCs, triggering DC inflammasome activation, secretion of IL-Iβ, and subsequent priming of interferon-γ (IFNγ) producing CD8⁺ T cells. (Ma, et al., Semin Immunol, 2010; 22: 113-24; Kroemer, et al, Bull Mem Acad R Med Belg, 2011; 166: 130-8, discussion 139-40). In some embodiments, the cumulative effects of the 3 arms of ICD and in particular CRT exposure (or the surface translocation of CRT) act to promote DC phagocytosis of tumor cells, thereby facilitating DC processing of tumor-expressed antigens and subsequent DC-associated cross-priming of CD8⁺ cytotoxic T lymphocytes (FIG. 2). (Bronte, V. Immunity, 2014; 41: 679-681).

Calreticulin, also known as calregulin, CRP55, CaBP3, calsequestrin-like protein, and endoplasmic reticulum resident protein 60 (ERp60), is a protein that in humans is encoded by the CALR gene. Calreticulin is a multifunctional protein that binds Ca²⁺ ions (a second messenger in signal transduction), rendering it inactive. In some instances, calreticulin is located in the lumen of the endoplasmic reticulum, where it interacts with misfolded proteins, inhibits their export from the endoplasmic reticulum into the Golgi apparatus and subsequently tags these misfolded proteins for degradation. In some cases, calreticulin further serves as a signaling ligand to recruit DCs to initiate phagocytosis.

In some embodiments, ICD is further sub-categorized into different types of ICD based on the ICD inducer. In some instances, an ICD inducer initiates the process of immunogenic cell death. In some cases, an ICD inducer comprises an agent that damages mitochondria resulting in the release of mtDNA. In some cases, an ICD inducer comprises micronuclei formed during cellular stress. In some cases, an ICD inducer comprises radiation. Exemplary types of radiation include UV radiation and γ radiation. In some cases, an ICD inducer comprises UV radiation. In some cases, an ICD inducer comprises γ radiation.

In other cases, an ICD inducer comprises a small molecule. In some cases, the small molecule comprises a chemotherapeutic agent. Exemplary chemotherapeutic agents include, but are not limited to, an anthracycline such as doxorubicin or mitoxantrone; a cyclophosphamide such as mafosfamide; bortezomib, daunorubicin, docetaxel, oxaliplatin or paclitaxel. In some instances, an ICD inducer comprises doxorubicin, mitoxantrone, mafosfamide, bortezomib, daunorubicin, docetaxel, oxaliplatin, paclitaxel, or any combinations thereof. In some instances, an ICD inducer comprises digitoxin or digoxin. In some instances, an ICD inducer comprises digitoxin. In some instances, an ICD inducer comprises digoxin. In some instances, an ICD inducer comprises septacidin. In some cases, an ICD inducer comprises a combination of cisplatin and thapsigargin. In some cases, an ICD inducer comprises a combination of cisplatin and tunicamycin.

In additional cases, an ICD inducer comprises a biologic. In such cases, a biologic comprises a protein or functional fragments thereof, a polypeptide, an oligosaccharide, a lipid, a nucleic acid (e.g., DNA or RNA) or a protein-payload conjugate. In some cases, a protein or functional fragments thereof comprises an enzyme, a glycoprotein, or a protein capable of inducing ICD. In some cases, a protein or functional fragments thereof comprises a humanized antibody or binding fragment thereof, a chimeric antibody or binding fragment thereof, a veneer antibody or binding fragment thereof, a monoclonal antibody or binding fragment thereof, a bispecific antibody or binding fragment thereof, an Fab, an Fab′, an F(ab′)₂, an F(ab′)₃, an scFv, an sc(Fv)₂, a dsFv, a diabody, a minibody, or a nanobody or binding fragments thereof. In some cases, a protein-payload conjugate comprises a protein or functional fragments thereof conjugated to a payload (e.g., a small molecule payload). In some cases, an exemplary protein-payload conjugate is trastuzumab emtansine.

In some embodiments, CRT exposure leads to phagocytosis by dendritic cells, leading to generating a population of cytosolic DNA. In some cases, cytosolic DNA sensor such as cyclic GMP-AMP synthase detects the presence of the cytosolic DNA and subsequently triggers inflammatory responses (e.g., generation of type I IFNs) via the STING-mediated pathway.

### Phosphodiesterases

In some embodiments, tumor cells circumvent the STING-mediated type I IFN production through overexpression of a phosphodiesterase. Phosphodiesterases comprise a class of enzymes that catalyze the hydrolysis of a phosphodiester bond. In some instances, this class comprises cyclic nucleotide phosphodiesterases, phospholipases C and D, autotaxin, sphingomyelin phosphodiesterase, DNases, RNases, restriction endonucleases, and small-molecule phosphodiesterases.

Cyclic nucleotide phosphodiesterases (PDEs) regulate the cyclic nucleotides cAMP and cGMP. In some instances, cAMP and cGMP function as intracellular second messengers to transduce a variety of extracellular signals including hormones, light, and neurotransmitters. In some cases, PDEs degrade cyclic nucleotides to their corresponding monophosphates, thereby regulating the intracellular concentrations of cyclic nucleotides and their effects on signal transduction.

In some embodiments, PDEs are classified into classes I, II and III. In some cases, mammalian PDEs, which belong to Class I PDEs, are further divided into 12 families (PDE1-PDE12) based on their substrate specificity and affinity, sensitivity to cofactors, and sensitivity to inhibitory agents. In some cases, the different families of mammalian PDEs further contain splice variants that can be unique in tissue-expression patterns, gene regulation, enzymatic regulation by phosphorylation and regulatory proteins, subcellular localization, and interaction with association proteins.

PDE1 family comprises Ca²⁺/calmodulin-dependent PDEs. In some cases, PDE1 is encoded by at least three different genes, each having at least two different splice variants, PDE1A and PDE1B. In some cases, PDE1 isozymes are regulated in vitro by phosphorylation/dephosphorylation. For example, phosphorylation decreases the affinity of PDE for calmodulin, decreases the activity of PDE1, and increases steady state levels of cAMP. In some cases, PDE1 is observed in the lung, heart, and brain.

PDE2s are cGMP-stimulated PDEs that have been observed in the cerebellum, neocortex, heart, kidney, lung, pulmonary artery, and skeletal muscle. In some cases, PDE2 mediates the effects of cAMP on catecholamine secretion, participate in the regulation of aldosterone, and play a role in olfactory signal transduction.

The family of PDE3s has a high affinity for both cGMP and cAMP. PDE3 plays a role in stimulating myocardial contractility, inhibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. In some instances, isozymes of PDE3 are regulated by cAMP-dependent protein kinase, or by insulin-dependent kinases.

In some embodiments, PDE4s are specific for cAMP and are activated by cAMP-dependent phosphorylation. In some cases, PDE4s are localized to airway smooth muscle, the vascular endothelium, and all inflammatory cells.

PDE5s exert selective recognition for cGMP as a substrate, and comprise two allosteric cGMP-specific binding sites. In some cases, binding of cGMP to these allosteric binding sites modulate phosphorylation of PDE5 by cGMP-dependent protein kinase. In some cases, elevated levels of PDE5 are found in vascular smooth muscle, platelets, lung, and kidney.

PDE6s, the photoreceptor cyclic nucleotide phosphodiesterases, are involved in the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which may further play a regulatory role in PDE6 function.

The PDE7 family of PDEs is cAMP specific and comprises one known member having multiple splice variants. Although mRNAs encoding PDE7s are found in skeletal muscle, heart, brain, lung, kidney, and pancreas, expression of PDE7 proteins is restricted to specific tissue types. Further, PDE7s shares a high degree of homology to the PDE4 family.

PDE8s are cAMP specific, and similar to PDE7, are closely related to the PDE4 family. In some cases, PDE8s are expressed in thyroid gland, testis, eye, liver, skeletal muscle, heart, kidney, ovary, and brain.

PDE9s are cGMP specific and closely resemble the PDE8 family of PDEs. In some cases, PDE9s are expressed in kidney, liver, lung, brain, spleen, and small intestine.

PDE10s are dual-substrate PDEs, hydrolyzing both cAMP and cGMP. In some instances, PDE10s are expressed in brain, thyroid, and testis.

PDE11s, similar to PDE10s, are dual-substrate PDEs that hydrolyze both cAMP and cGMP. In some instances, PDE11s are expressed in the skeletal muscle, brain, lung, spleen, prostate gland, and testis.

PDE12s hydrolyze cAMP and oligoadenylates (e.g., 2′,5′-oligoadenylate). In some cases, although PDE12 hydrolyzes the 2′5′ linkage, PDE12 does not exhibit activity toward 2′3′-cGAMP.

### Ecto-Nucleotide Pyrophosphatase/Phosphodiesterase

In some embodiments, the class of phosphodiesterases also comprises an ecto-nucleotide pyrophosphatase/phosphodiesterase. Ecto-nucleotide pyrophosphatase/phosphodiesterases (ENPP) or nucleotide pyrophosphatase/phosphodiesterases (NPP) are a subfamily of ectonucleotidases which hydrolyze the pyrophosphate and phosphodiester bonds of their substrates to nucleoside 5′-monophosphates. In some embodiments, ENPP (or NPP) comprises seven members, ENPP-1, ENPP-2, ENPP-3, ENPP-4, ENPP-5, ENPP-6 and ENPP-7.

The ecto-nucleotide pyrophosphatase/phosphodiesterase 1 (ENPP-1) protein (also known as PC-1) is a type II transmembrane glycoprotein comprising two identical disulfide-bonded subunits. In some instances, ENPP-1 is expressed in precursor cells and promotes osteoblast differentiation and regulates bone mineralization. In some instances, ENPP-1 negatively regulates bone mineralization by hydrolyzing extracellular nucleotide triphosphates (NTPs) to produce inorganic pyrophosphate (PPi). In some cases, expression of ENPP-1 has been observed in pancreas, kidney, bladder, and the liver. In some cases, ENPP-1 has been observed to be overexpressed in cancer cells, e.g., in breast cancer cells and glioblastoma cells.

In some embodiments, ENPP-1 has a broad specificity and cleaves a variety of substrates, including phosphodiester bonds of nucleotides and nucleotide sugars and pyrophosphate bonds of nucleotides and nucleotide sugars. In some instances, ENPP-1 functions to hydrolyze nucleoside 5′ triphosphates to their corresponding monophosphates and also hydrolyze diadenosine polyphosphates. In some cases, ENPP-1 hydrolyzes the 2′5′ linkage of cyclic nucleotides. In some cases, ENPP-1 degrades 2′3′-cGAMP, a substrate of STING.

In some embodiments, ENPP-1 comprises two N-terminal somatomedin B (SMB)-like domains (SMB1 and SMB2), a catalytic domain and a C-terminal nuclease-like domain. In some cases, the two SMB domains is connected to the catalytic domain by a first flexible linker, while the catalytic domain is further connected to the nuclease-like domain by a second flexible linker. In some instances, the SMB domains facilitate ENPP-1 dimerization. In some cases, the catalytic domain comprises the NTP binding site. In some cases, the nuclease-like domain comprises an EF-hand motif, which binds Ca⁺² ion.

In some cases, ENPP-2 and ENPP-3 are type II transmembrane glycoproteins that share a similar architecture with ENPP-1, for example, comprising the two N-terminal SMB-like domains, a catalytic domain, and a nuclease-like domain. In some instances, ENPP-2 hydrolyzes lysophospholipids to produce lysophosphatidic acid (LPA) or sphingosylphosphorylcholine (SPC) to produce sphingosine-1 phosphate (S1P). In some cases, ENPP-3 is identified to regulate N-acetylglucosaminyltransferase GnT-IX (GnT-Vb).

In some embodiments, ENPP-4-ENPP-7 are shorter proteins compared to ENPP-1-ENPP-3 and comprise a catalytic domain and lack the SMB-like and nuclease-like domains. ENPP-6 is a choline-specific glycerophosphodiesterase, with lysophospholipase C activity towards lysophosphatidylcholine (LPC). ENPP-7 is an alkaline sphingomyelinase (alk-SMase) with no detectable nucleotidase activity.

Inhibitor of 2′3′-cGAMP Degradation Polypeptide

In some embodiments, disclosed herein include an inhibitor of a 2′3′-cGAMP degradation polypeptide. In some instances, a 2′3′-cGAMP degradation polypeptide comprises a PDE protein. In some instances, a 2′3′-cGAMP degradation polypeptide comprises a PDE5 protein. In some instances, a 2′3′-cGAMP degradation polypeptide comprises a PDE10 protein. In some instances, a 2′3′-cGAMP degradation polypeptide comprises a Pan-PDE protein. In some cases, a 2′3′-cGAMP degradation polypeptide comprises a ENPP-1 protein. In some cases, an inhibitor of a 2′3′-cGAMP degradation polypeptide is a small molecule inhibitor. In some cases, an inhibitor of a 2′3′-cGAMP degradation polypeptide comprises a PDE5 inhibitor. In some cases, an inhibitor of a 2′3′-cGAMP degradation polypeptide comprises a PDE10 inhibitor. In some cases, an inhibitor of a 2′3′-cGAMP degradation polypeptide comprises a Pan-PDE inhibitor. In some cases, an inhibitor of a 2′3′-cGAMP degradation polypeptide comprises an ENPP-1 inhibitor.

In some embodiments, an inhibitor of a 2′3′-cGAMP degradation polypeptide (e.g., a ENPP-1 inhibitor) described herein is a reversible inhibitor. Reversible inhibitor interacts with an enzyme with non-covalent interactions, e.g., hydrogen bonds, hydrophobic interactions, and/or ionic bonds. In some instances, a reversible inhibitor is further classified as a competitive inhibitor, an allosteric inhibitor or a mixed inhibitor. In competitive inhibition, both the inhibitor and the substrate compete for the same active site. In allosteric inhibition, the inhibitor binds to the enzyme at a non-active site which modulates the enzyme's activity but does not affect binding of the substrate. In some cases, an inhibitor of a 2′3′-cGAMP degradation polypeptide (e.g., a ENPP-1 inhibitor) described herein is a competitive inhibitor. In other cases, an inhibitor of a 2′3′-cGAMP degradation polypeptide (e.g., a ENPP-1 inhibitor) described herein is an allosteric inhibitor. In some cases, an inhibitor of a 2′3′-cGAMP degradation polypeptide (e.g., a ENPP-1 inhibitor) described herein is a mixed inhibitor. In some instances, a ENPP-1 inhibitor described herein is a competitive inhibitor. In other instances, a ENPP-1 inhibitor described herein is an allosteric inhibitor. In other instances, a ENPP-1 inhibitor described herein is a mixed inhibitor.

In some embodiments, an inhibitor of a 2′3′-cGAMP degradation polypeptide (e.g., a ENPP-1 inhibitor) described herein is an irreversible inhibitor. Irreversible inhibitor interacts with an enzyme with covalent interaction. In some cases, a ENPP-1 is an irreversible inhibitor.

In some embodiments, an inhibitor of a 2′3′-cGAMP degradation polypeptide (e.g., a ENPP-1 inhibitor) binds to one or more domains of a PDE described herein. In some cases, a PDE inhibitor binds to one or more domains of ENPP-1. As described above, ENPP-1 comprises a catalytic domain and a nuclease-like domain. In some instances, an inhibitor of a 2′3′-cGAMP degradation polypeptide (e.g., a ENPP-1 inhibitor) binds to the catalytic domain of ENPP-1. In some cases, an inhibitor of a 2′3′-cGAMP degradation polypeptide (e.g., a ENPP-1 inhibitor) binds to the nuclease-like domain of ENPP-1.

In some cases, an inhibitor of a 2′3′-cGAMP degradation polypeptide (e.g., a ENPP-1 inhibitor) selectively binds to a region on PDE (e.g., ENPP-1) also recognized by GMP. In some cases, an inhibitor of a 2′3′-cGAMP degradation polypeptide (e.g., a ENPP-1 inhibitor) selectively binds to a region on PDE (e.g., ENPP-1) also recognized by GMP but interacts weakly with the region that is bound by AMP. In some instances, an inhibitor of a 2′3′-cGAMP degradation polypeptide (e.g., a ENPP-1 inhibitor) does not inhibit the ATP hydrolysis function of PDE.

In some embodiments, an inhibitor of a 2′3′-cGAMP degradation polypeptide (e.g., a ENPP-1 inhibitor) comprises a di-adenosine pentaphosphate analogue, an ATP analogue, an oxadiazole derivative, a biscoumarine derivative, or a combination. In some instances, an inhibitor of a 2′3′-cGAMP degradation polypeptide (e.g., a ENPP-1 inhibitor) comprises a compound, its analogue, or its derivative as illustrated in Scheme I.

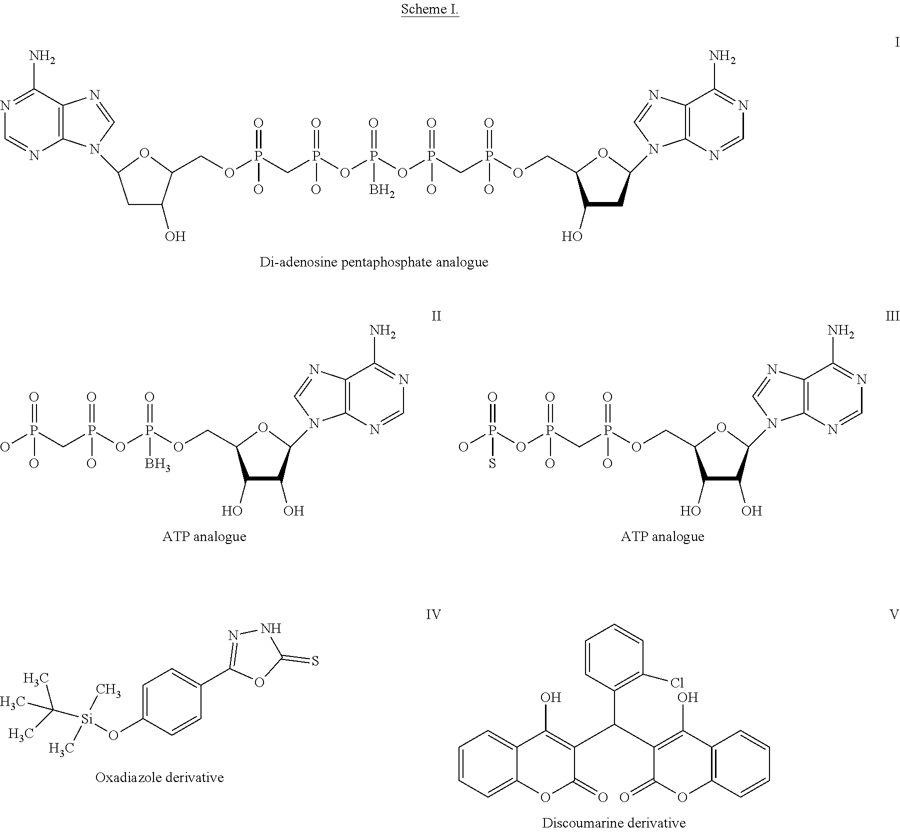

In some embodiments, an inhibitor of a 2′3′-cGAMP degradation polypeptide (e.g., a ENPP-1 inhibitor) comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, an inhibitor of a 2′3′-cGAMP degradation polypeptide (e.g., a ENPP-1 inhibitor) is ARL67156:

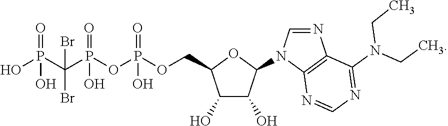 [SMILES: CCN(CC)c1ncnc2c1ncn2[C@@H]1O[C@H](COP(=O)(O)OP(=O)(O)C(Br)(Br)P(=O)(O)O)[C@@H](O)[C@H]1O]

In some instances, an inhibitor of a 2′3′-cGAMP degradation polypeptide (e.g., a ENPP-1 inhibitor) is diadenosine 5′,5″-boranopolyphosphonate:

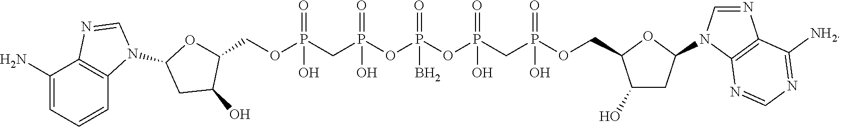

In some instances, an inhibitor of a 2′3′-cGAMP degradation polypeptide (e.g., a ENPP-1 inhibitor) is adenosine 5′-(α-borano)-β,γ-methylene triphosphate:

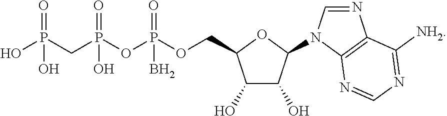

In some instances, an inhibitor of a 2′3′-cGAMP degradation polypeptide (e.g., a ENPP-1 inhibitor) is adenosine 5′-(γ-thio)-α,β-methylene triphosphate:

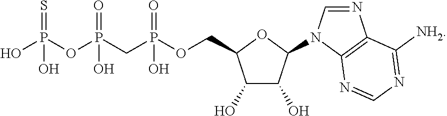 [SMILES: Nc1ncnc2c1ncn2[C@@H]1O[C@H](COP(=O)(O)CP(=O)(O)OP(O)(O)=S)[C@@H](O)[C@H]1O]

In some instances, an inhibitor of a 2′3′-cGAMP degradation polypeptide (e.g., a ENPP-1 inhibitor) is an oxadiazole derivative:

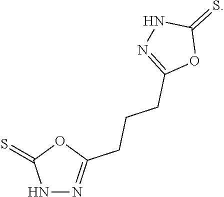 [SMILES: S=c1[nH]nc(CCCc2n[nH]c(=S)o2)o1]

In some instances, an inhibitor of a 2′3′-cGAMP degradation polypeptide (e.g., a ENPP-1 inhibitor) is a biscoumarine derivative:

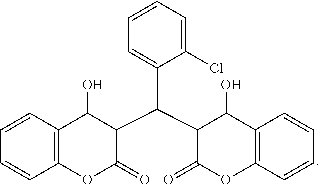 [SMILES: O=C1Oc2ccccc2C(O)C1C(c1ccccc1Cl)C1C(=O)Oc2ccccc2C1O]

In some instances, an inhibitor of a 2′3′-cGAMP degradation polypeptide (e.g., a ENPP-1 inhibitor) is reactive blue 2:

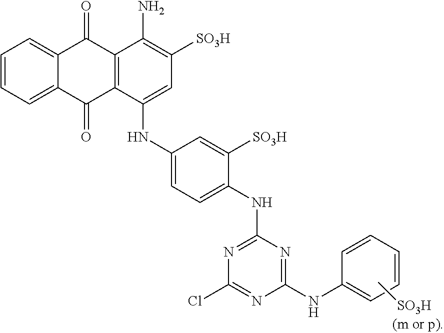

In some instances, an inhibitor of a 2′3′-cGAMP degradation polypeptide (e.g., a ENPP-1 inhibitor) is suramin:

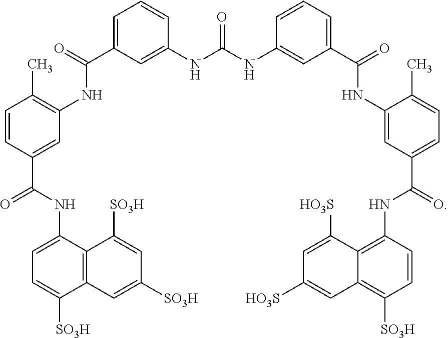 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

In some instances, an inhibitor of a 2′3′-cGAMP degradation polypeptide (e.g., a ENPP-1 inhibitor) is a quinazoline-4-piperidine-4-ethylsulfamide derivative:

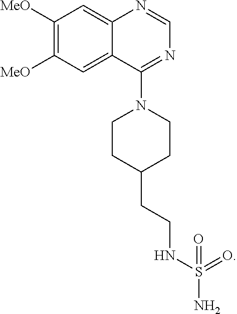 [SMILES: COc1cc2ncnc(N3CCC(CCNS(N)(=O)=O)CC3)c2cc1OC]

In some instances, an inhibitor of a 2′3′-cGAMP degradation polypeptide (e.g., a ENPP-1 inhibitor) is a thioacetamide derivative:

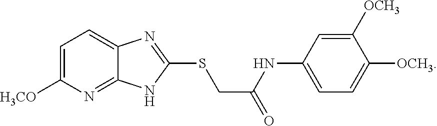 [SMILES: COc1ccc2nc(SCC(=O)Nc3ccc(OC)c(OC)c3)[nH]c2n1]

In some instances, an inhibitor of a 2′3′-cGAMP degradation polypeptide (e.g., a ENPP-1 inhibitor) is PSB-POM141:
[symbol] (a Keggin-type inorganic complex)

In some embodiments, an inhibitor of a 2′3′-cGAMP degradation polypeptide (e.g., a ENPP-1 inhibitor) comprises 2-(3H-imidazo[4,5-b]pyridin-2-ylthio)-N-(3,4-dimethoxyphenyl)acetamide or a derivative, analog, or salt thereof.

In some embodiments, an inhibitor of a 2′3′-cGAMP degradation polypeptide (e.g., a ENPP-1 inhibitor) comprises 2-(6-Amino-9H-purin-8-ylthio)-N-(3,4-dimethoxyphenyl)-acetamide, or a salt thereof.

In some embodiments, an inhibitor of a 2′3′-cGAMP degradation polypeptide (e.g., a ENPP-1 inhibitor) comprises N-(3,4-Dimethoxyphenyl)-2-(5-methoxy-3H-imidazo[4,5-b]-pyridin-2-ylthio)acetamide or a salt thereof.

In some embodiments, an inhibitor of a 2′3′-cGAMP degradation polypeptide (e.g., a ENPP-1 inhibitor) comprises 2-(1-(6,7-Dimethoxyquinazolin-4-yl)piperidin-4-yl)ethyl sulfamide or a salt thereof.

In some embodiments, an inhibitor of a 2′3′-cGAMP degradation polypeptide (e.g., a ENPP-1 inhibitor) comprises ((1-(6,7-Dimethoxyquinazolin-4-yl)piperidin-4-yl)methyl)sulfamide or a salt thereof.

In some embodiments, an inhibitor of a 2′3′-cGAMP degradation polypeptide (e.g., a ENPP-1 inhibitor) comprises SK4A (SAT0037) or a derivative or salt thereof.

In some instances, an inhibitor of a 2′3′-cGAMP degradation polypeptide (e.g., a ENPP-1 inhibitor) comprises a PDE inhibitor described in Chang, et al., “Imidazopyridine- and purine-thioacetamide derivatives: potent inhibitors of nucleotide pyrophosphatase/phosphodiesterase I (NPP1),” J. of Med. Chem., 57:10080-10100 (2014).

In some embodiments, an inhibitor of a 2′3′-cGAMP degradation polypeptide (e.g., a ENPP-1 inhibitor) comprises a PDE inhibitor described in Lee, et al., “Thiazolo[3,2-a]benzimidazol-3(2H)-one derivatives: structure-activity relationships of selective nucleotide pyrophosphatase/phosphodiesterase 1 (NPP1) inhibitors,” Bioorganic & Medicinal Chemistry, 24:3157-3165 (2016).

In some embodiments, an inhibitor of a 2′3′-cGAMP degradation polypeptide (e.g., a ENPP-1 inhibitor) comprises a PDE inhibitor described in Shayhidin, et al., “Quinazoline-4-piperidine sulfamides are specific inhibitors of human NPP1 and prevent pathological mineralization of valve interstitial cells,” British Journal of Pharmacology, 172:4189-4199 (2015).

In some embodiments, an inhibitor of a 2′3′-cGAMP degradation polypeptide (e.g., a ENPP-1 inhibitor) comprises a PDE inhibitor described in Li, et al., “Hydrolysis of 2′3′-cGAMP by ENPP-1 and design of nonhydrolyzable analogs,” Nature Chemical Biology, 10:1043-1048 (2014).

In some embodiments, an inhibitor of a 2′3′-cGAMP degradation polypeptide (e.g., a ENPP-1 inhibitor) comprises Compound 1:

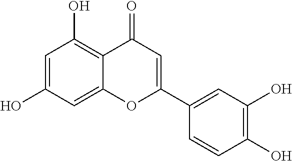 [SMILES: O=c1cc(-c2ccc(O)c(O)c2)oc2cc(O)cc(O)c12]

or a derivative, analog, or salt thereof.

In some embodiments, an inhibitor of a 2′3′-cGAMP degradation polypeptide (e.g., a ENPP-1 inhibitor) comprises Compound 2:

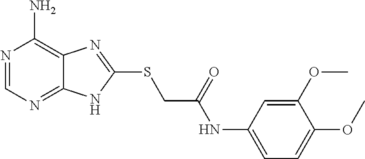 [SMILES: COc1ccc(NC(=O)CSc2nc3c(N)ncnc3[nH]2)cc1OC]

or a derivative, analog, or salt thereof.

In some embodiments, an inhibitor of a 2′3′-cGAMP degradation polypeptide (e.g., a ENPP-1 inhibitor) comprises Compound 3:

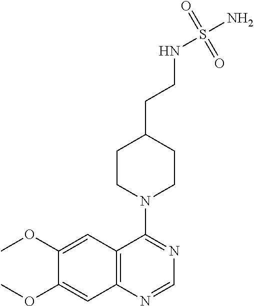 [SMILES: COc1cc2ncnc(N3CCC(CCNS(N)(=O)=O)CC3)c2cc1OC]

or a derivative, analog, or salt thereof.

### Methods of Use

In some embodiments, disclosed herein are methods of treating a subject having cancer. In some instances, the cancer is primed with an immunogenic cell death (ICD) inducer. In other instances, the cancer is treated with a PDE inhibitor prior to administering an ICD inducer or is treated simultaneously with a PDE inhibitor and an ICD inducer. In some cases, a method disclosed herein comprises treating a subject having a cancer primed by an ICD inducer by administering to the subject a phosphodiesterase (PDE) inhibitor, wherein the PDE inhibitor prevents hydrolysis of 2′3′-cGAMP. In some cases, a method disclosed herein comprises treating a subject having a cancer by administering to the subject a phosphodiesterase (PDE) inhibitor, wherein the PDE inhibitor prevents hydrolysis of 2′3′-cGAMP, and wherein the PDE inhibitor is administered either prior to administering the ICD inducer or simultaneously with the ICD inducer.

In some embodiments, a PDE comprises a cyclic nucleotide phosphodiesterase described supra. In some embodiments, the PDE comprises a PDE5 protein. In some cases, the PDE comprises a PDE10 protein. In some cases, the PDE comprises a Pan-PDE protein. In some embodiments, the PDE comprises an ectonucleotide pyrophosphatase/phosphodiesterase (ENPP) protein. In some cases, the ENPP protein comprises ectonucleotide pyrophosphatase/phosphodiesterase family member 1 (ENPP-1).

In some instances, a PDE inhibitor described herein comprises a small molecule. In some cases, the PDE inhibitor is a PDE5 inhibitor. In some cases, the PDE inhibitor is a PDE10 inhibitor. In some cases, the PDE inhibitor is a Pan-PDE inhibitor. In some cases, the PDE inhibitor is an ENPP-1 inhibitor.

In some embodiments, a PDE inhibitor described herein is a reversible inhibitor. In some instances, a reversible inhibitor is further classified as a competitive inhibitor or an allosteric inhibitor. In some cases, a PDE inhibitor described herein is a competitive inhibitor. In other cases, a PDE inhibitor described herein is an allosteric inhibitor. In some cases, a PDE inhibitor described herein is a mixed inhibitor. In some instances, a ENPP-1 inhibitor described herein is a competitive inhibitor. In other instances, a ENPP-1 inhibitor described herein is an allosteric inhibitor. In some instances, a ENPP-1 inhibitor described herein is a mixed inhibitor.

In some embodiments, a PDE inhibitor described herein is an irreversible inhibitor. In some cases, a ENPP-1 is an irreversible inhibitor.

In some embodiments, a PDE inhibitor binds to one or more domains of a PDE described herein. In some cases, a PDE inhibitor binds to one or more domains of ENPP-1. As described above, ENPP-1 comprises a catalytic domain and a nuclease-like domain. In some instances, a PDE inhibitor binds to the catalytic domain of ENPP-1. In some cases, a PDE inhibitor binds to the nuclease-like domain of ENPP-1.

In some cases, a PDE inhibitor selectively binds to a region on PDE (e.g., ENPP-1) also recognized by GMP. In some cases, a PDE inhibitor selectively binds to a region on PDE (e.g., ENPP-1) also recognized by GMP but interacts weakly with the region that is bound by AMP.

In some embodiments, a PDE inhibitor comprises a di-adenosine pentaphosphate analogue, an ATP analogue, an oxadiazole derivative, a biscoumarine derivative, or a combination. In some instances, a PDE inhibitor comprises a compound, its analogue, or its derivative as illustrated in Scheme I.

In some embodiments, a PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some instances, a PDE inhibitor is ARL67156. In some instances, a PDE inhibitor is diadenosine 5′,5″-boranopolyphosphonate. In some instances, a PDE inhibitor is adenosine 5′-(α-borano)-β,γ-methylene triphosphate. In some instances, a PDE inhibitor is adenosine 5′-(γ-thio)-α,β-methylene triphosphate. In some instances, a PDE inhibitor is an oxadiazole derivative. In some instances, a PDE inhibitor is a biscoumarine derivative. In some instances, a PDE inhibitor is reactive blue 2. In some instances, a PDE inhibitor is suramin. In some instances, a PDE inhibitor is a quinazoline-4-piperidine-4-ethylsulfamide derivative. In some instances, a PDE inhibitor is a thioacetamide derivative. In some instances, a PDE inhibitor is PSB-POM141 (a Keggin-type inorganic complex).

In some embodiments, a PDE inhibitor comprises 2-(3H-imidazo[4,5-b]pyridin-2-ylthio)-N-(3,4-dimethoxyphenyl)acetamide or a derivative, analog, or salt thereof.

In some embodiments, a PDE inhibitor comprises 2-(6-Amino-9H-purin-8-ylthio)-N-(3,4-dimethoxyphenyl)-acetamide, or a salt thereof.

In some embodiments, a PDE inhibitor comprises N-(3,4-Dimethoxyphenyl)-2-(5-methoxy-3H-imidazo[4,5-b]-pyridin-2-ylthio)acetamide or a salt thereof.

In some embodiments, a PDE inhibitor comprises 2-(1-(6,7-Dimethoxyquinazolin-4-yl)piperidin-4-yl)ethyl sulfamide or a salt thereof.

In some embodiments, a PDE inhibitor comprises ((1-(6,7-Dimethoxyquinazolin-4-yl)piperidin-4-yl)methyl)sulfamide or a salt thereof.

In some embodiments, a PDE inhibitor comprises SK4A (SAT0037) or a derivative or salt thereof.

In some instances, a PDE inhibitor comprises a PDE inhibitor described in Chang, et al., “Imidazopyridine- and purine-thioacetamide derivatives: potent inhibitors of nucleotide pyrophosphatase/phosphodiesterase I (NPP1),” J. of Med. Chem., 57:10080-10100 (2014).

In some embodiments, a PDE inhibitor comprises a PDE inhibitor described in Lee, et al., “Thiazolo[3,2-a]benzimidazol-3(2H)-one derivatives: structure-activity relationships of selective nucleotide pyrophosphatase/phosphodiesterase 1 (NPP1) inhibitors,” Bioorganic & Medicinal Chemistry, 24:3157-3165 (2016).

In some embodiments, a PDE inhibitor comprises a PDE inhibitor described in Shayhidin, et al., “Quinazoline-4-piperidine sulfamides are specific inhibitors of human NPP1 and prevent pathological mineralization of valve interstitial cells,” British Journal of Pharmacology, 172:4189-4199 (2015).

In some embodiments, a PDE inhibitor comprises a PDE inhibitor described in Li, et al., “Hydrolysis of 2′3′-cGAMP by ENPP-1 and design of nonhydrolyzable analogs,” Nature Chemical Biology, 10:1043-1048 (2014).

In some embodiments, a PDE inhibitor comprises Compound 1:

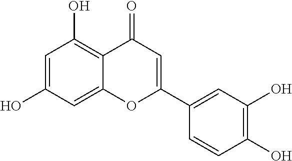 [SMILES: O=c1cc(-c2ccc(O)c(O)c2)oc2cc(O)cc(O)c12]

or a derivative, analog, or salt thereof.

In some embodiments, a PDE inhibitor comprises Compound 2:

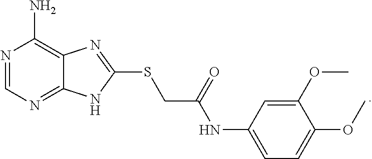 [SMILES: COc1ccc(NC(=O)CSc2nc3c(N)ncnc3[nH]2)cc1OC]

or a derivative, analog, or salt thereof.

In some embodiments, a PDE inhibitor comprises Compound 3:

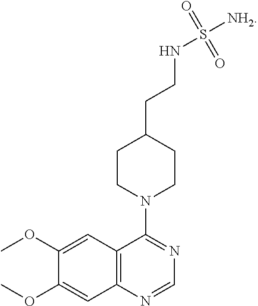 [SMILES: COc1cc2ncnc(N3CCC(CCNS(N)(=O)=O)CC3)c2cc1OC]

or a derivative, analog, or salt thereof.

In some embodiments, a cancer described herein is a solid tumor. Solid tumor comprises neoplasms and lesions derived from cells other than blood, bone marrow, or lymphatic cells. In some cases, exemplary solid tumors include breast cancer and lung cancer.

In some embodiments, a cancer described herein is a hematologic malignancy. A hematologic malignancy comprises an abnormal cell growth of blood, bone marrow, and/or lymphatic cells. For instances, an exemplary hematologic malignancy comprises multiple myeloma. In some instances, a hematologic malignancy is a leukemia, a lymphoma or a myeloma. In some cases, a hematologic malignancy is a B-cell malignancy.

In some embodiments, a cancer described herein is a relapsed or refractory cancer.

In some embodiments, a cancer described herein is a metastatic cancer.

In some cases, an ICD inducer comprises an agent that damages mitochondria resulting in the release of mtDNA. In some cases, an ICD inducer comprises an agent that induces micronuclei formation.

In some embodiments, an ICD inducer comprises radiation. In some cases, the radiation comprises UV radiation. In other cases, the radiation comprises γ radiation.

In some embodiments, an ICD inducer comprises a small molecule compound or a biologic. As described above, an ICD small molecule inducer optionally comprises a chemotherapeutic agent. In some cases, the chemotherapeutic agent comprises an anthracycline. In some cases, the anthracycline is doxorubicin or mitoxantrone. In some instances, the chemotherapeutic agent comprises a cyclophosphamide. In some instances, the cyclophosphamide is mafosfamide. In some embodiments, the chemotherapeutic agent is selected from bortezomib, daunorubicin, docetaxel, oxaliplatin, paclitaxel, or a combination thereof. In some cases, the ICD inducer comprises digitoxin or digoxin. In some cases, the ICD inducer comprises septacidin. In some cases, the ICD inducer comprises a combination of cisplatin and thapsigargin. In some cases, the ICD inducer comprises a combination of cisplatin and tunicamycin.

In some embodiments, an ICD inducer comprises a biologic (e.g., a protein-payload conjugate such as trastuzumab emtansine). In some cases, the ICD inducer comprises an activator of calreticulin (CRT) exposure.

In some embodiments, a PDE inhibitor is administered to a subject at least 0.5, 1, 1.5, 2, 3, 4, 5, 6, 7, 8, 9, 10, 11, 12, 18, 24, 36 or 48 hours after administration of the ICD inducer. In some cases, the PDE inhibitor is administered to the subject at least 0.5 hour after administration of the ICD inducer. In some cases, the PDE inhibitor is administered to the subject at least 1 hour after administration of the ICD inducer. In some cases, the PDE inhibitor is administered to the subject at least 1.5 hours after administration of the ICD inducer. In some cases, the PDE inhibitor is administered to the subject at least 2 hours after administration of the ICD inducer. In some cases, the PDE inhibitor is administered to the subject at least 3 hours after administration of the ICD inducer. In some cases, the PDE inhibitor is administered to the subject at least 4 hours after administration of the ICD inducer. In some cases, the PDE inhibitor is administered to the subject at least 5 hours after administration of the ICD inducer. In some cases, the PDE inhibitor is administered to the subject at least 6 hours after administration of the ICD inducer. In some cases, the PDE inhibitor is administered to the subject at least 7 hours after administration of the ICD inducer. In some cases, the PDE inhibitor is administered to the subject at least 8 hours after administration of the ICD inducer. In some cases, the PDE inhibitor is administered to the subject at least 9 hours after administration of the ICD inducer. In some cases, the PDE inhibitor is administered to the subject at least 10 hours after administration of the ICD inducer. In some cases, the PDE inhibitor is administered to the subject at least 11 hours after administration of the ICD inducer. In some cases, the PDE inhibitor is administered to the subject at least 12 hours after administration of the ICD inducer. In some cases, the PDE inhibitor is administered to the subject at least 18 hours after administration of the ICD inducer. In some cases, the PDE inhibitor is administered to the subject at least 24 hours after administration of the ICD inducer. In some cases, the PDE inhibitor is administered to the subject at least 36 hours after administration of the ICD inducer. In some cases, the PDE inhibitor is administered to the subject at least 48 hours after administration of the ICD inducer.

In some embodiments, a PDE inhibitor is administered to a subject at least 1, 2, 3, 4, 5, 6, 7, 8, 9, 10, 11, 12, 13, 14, 28, 30 or 40 days after administration of the ICD inducer. In some cases, the PDE inhibitor is administered to the subject at least 1 day after administration of the ICD inducer. In some cases, the PDE inhibitor is administered to the subject at least 2 days after administration of the ICD inducer. In some cases, the PDE inhibitor is administered to the subject at least 3 days after administration of the ICD inducer. In some cases, the PDE inhibitor is administered to the subject at least 4 days after administration of the ICD inducer. In some cases, the PDE inhibitor is administered to the subject 4 days after administration of the ICD inducer. In some cases, the PDE inhibitor is administered to the subject at least 5 days after administration of the ICD inducer. In some cases, the PDE inhibitor is administered to the subject at least 6 days after administration of the ICD inducer. In some cases, the PDE inhibitor is administered to the subject at least 7 days after administration of the ICD inducer. In some cases, the PDE inhibitor is administered to the subject at least 8 days after administration of the ICD inducer. In some cases, the PDE inhibitor is administered to the subject at least 9 days after administration of the ICD inducer. In some cases, the PDE inhibitor is administered to the subject at least 10 days after administration of the ICD inducer. In some cases, the PDE inhibitor is administered to the subject at least 11 days after administration of the ICD inducer. In some cases, the PDE inhibitor is administered to the subject at least 12 days after administration of the ICD inducer. In some cases, the PDE inhibitor is administered to the subject at least 13 days after administration of the ICD inducer. In some cases, the PDE inhibitor is administered to the subject at least 14 days after administration of the ICD inducer. In some cases, the PDE inhibitor is administered to the subject at least 28 days after administration of the ICD inducer. In some cases, the PDE inhibitor is administered to the subject at least 30 days after administration of the ICD inducer.

In some embodiments, a PDE inhibitor is administered to a subject at least 0.5, 1, 1.5, 2, 3, 4, 5, 6, 7, 8, 9, 10, 11, 12, 18, 24, 36 or 48 hours prior to administration of the ICD inducer. In some cases, the PDE inhibitor is administered to the subject at least 0.5 hour prior to administration of the ICD inducer. In some cases, the PDE inhibitor is administered to the subject at least 1 hour prior to administration of the ICD inducer. In some cases, the PDE inhibitor is administered to the subject at least 1.5 hours prior to administration of the ICD inducer. In some cases, the PDE inhibitor is administered to the subject at least 2 hours prior to administration of the ICD inducer. In some cases, the PDE inhibitor is administered to the subject at least 3 hours prior to administration of the ICD inducer. In some cases, the PDE inhibitor is administered to the subject at least 4 hours prior to administration of the ICD inducer. In some cases, the PDE inhibitor is administered to the subject at least 5 hours prior to administration of the ICD inducer. In some cases, the PDE inhibitor is administered to the subject at least 6 hours prior to administration of the ICD inducer. In some cases, the PDE inhibitor is administered to the subject at least 7 hours prior to administration of the ICD inducer. In some cases, the PDE inhibitor is administered to the subject at least 8 hours prior to administration of the ICD inducer. In some cases, the PDE inhibitor is administered to the subject at least 9 hours prior to administration of the ICD inducer. In some cases, the PDE inhibitor is administered to the subject at least 10 hours prior to administration of the ICD inducer. In some cases, the PDE inhibitor is administered to the subject at least 11 hours prior to administration of the ICD inducer. In some cases, the PDE inhibitor is administered to the subject at least 12 hours prior to administration of the ICD inducer. In some cases, the PDE inhibitor is administered to the subject at least 18 hours prior to administration of the ICD inducer. In some cases, the PDE inhibitor is administered to the subject at least 24 hours prior to administration of the ICD inducer. In some cases, the PDE inhibitor is administered to the subject at least 36 hours prior to administration of the ICD inducer. In some cases, the PDE inhibitor is administered to the subject at least 48 hours prior to administration of the ICD inducer.

In some embodiments, a PDE inhibitor is administered to a subject at least 1, 2, 3, 4, 5, 6, 7, 8, 9, 10, 11, 12, 13, 14, 28, 30 or 40 days prior to administration of the ICD inducer. In some cases, the PDE inhibitor is administered to the subject at least 1 day prior to administration of the ICD inducer. In some cases, the PDE inhibitor is administered to the subject at least 2 days prior to administration of the ICD inducer. In some cases, the PDE inhibitor is administered to the subject at least 3 days prior to administration of the ICD inducer. In some cases, the PDE inhibitor is administered to the subject at least 4 days prior to administration of the ICD inducer. In some cases, the PDE inhibitor is administered to the subject at least 5 days prior to administration of the ICD inducer. In some cases, the PDE inhibitor is administered to the subject at least 6 days prior to administration of the ICD inducer. In some cases, the PDE inhibitor is administered to the subject at least 7 days prior to administration of the ICD inducer. In some cases, the PDE inhibitor is administered to the subject at least 8 days prior to administration of the ICD inducer. In some cases, the PDE inhibitor is administered to the subject at least 9 days prior to administration of the ICD inducer. In some cases, the PDE inhibitor is administered to the subject at least 10 days prior to administration of the ICD inducer. In some cases, the PDE inhibitor is administered to the subject at least 11 days prior to administration of the ICD inducer. In some cases, the PDE inhibitor is administered to the subject at least 12 days prior to administration of the ICD inducer. In some cases, the PDE inhibitor is administered to the subject at least 13 days prior to administration of the ICD inducer. In some cases, the PDE inhibitor is administered to the subject at least 14 days prior to administration of the ICD inducer. In some cases, the PDE inhibitor is administered to the subject at least 28 days prior to administration of the ICD inducer. In some cases, the PDE inhibitor is administered to the subject at least 30 days prior to administration of the ICD inducer.

In some cases, a PDE inhibitor is administered simultaneously with an ICD inducer.

In some cases, a PDE inhibitor is administered continuously for 1, 2, 3, 4, 5, 6, 7, 8, 9, 10, 14, 15, 28, 30 or more days. In some instances, the PDE inhibitor is administered continuously for 1 or more days. In some instances, the PDE inhibitor is administered continuously for 2 or more days. In some instances, the PDE inhibitor is administered continuously for 3 or more days. In some instances, the PDE inhibitor is administered continuously for 4 or more days. In some instances, the PDE inhibitor is administered continuously for 5 or more days. In some instances, the PDE inhibitor is administered continuously for 6 or more days. In some instances, the PDE inhibitor is administered continuously for 7 or more days. In some instances, the PDE inhibitor is administered continuously for 8 or more days. In some instances, the PDE inhibitor is administered continuously for 9 or more days. In some instances, the PDE inhibitor is administered continuously for 10 or more days. In some instances, the PDE inhibitor is administered continuously for 14 or more days. In some instances, the PDE inhibitor is administered continuously for 15 or more days. In some instances, the PDE inhibitor is administered continuously for 28 or more days. In some instances, the PDE inhibitor is administered continuously for 30 or more days.

In some cases, a PDE inhibitor is administered at predetermined time intervals for 1, 2, 3, 4, 5, 6, 7, 8, 9, 10, 14, 15, 28, 30 or more days. In some instances, the PDE inhibitor is administered at predetermined time intervals for 1 or more days. In some instances, the PDE inhibitor is administered at predetermined time intervals for 2 or more days. In some instances, the PDE inhibitor is administered at predetermined time intervals for 3 or more days. In some instances, the PDE inhibitor is administered at predetermined time intervals for 4 or more days. In some instances, the PDE inhibitor is administered at predetermined time intervals for 5 or more days. In some instances, the PDE inhibitor is administered at predetermined time intervals for 6 or more days. In some instances, the PDE inhibitor is administered at predetermined time intervals for 7 or more days. In some instances, the PDE inhibitor is administered at predetermined time intervals for 8 or more days. In some instances, the PDE inhibitor is administered at predetermined time intervals for 9 or more days. In some instances, the PDE inhibitor is administered at predetermined time intervals for 10 or more days. In some instances, the PDE inhibitor is administered at predetermined time intervals for 14 or more days. In some instances, the PDE inhibitor is administered at predetermined time intervals for 15 or more days. In some instances, the PDE inhibitor is administered at predetermined time intervals for 28 or more days. In some instances, the PDE inhibitor is administered at predetermined time intervals for 30 or more days.

In some embodiments, a PDE inhibitor is administered at predetermined time intervals for 1, 2, 3, 4, 5, 6, 7, 8, 9, 10, 11, 12, 24, 36 or more months. In some instances, the PDE inhibitor is administered at predetermined time intervals for 1 or more month. In some instances, the PDE inhibitor is administered at predetermined time intervals for 2 or more months. In some instances, the PDE inhibitor is administered at predetermined time intervals for 3 or more months. In some instances, the PDE inhibitor is administered at predetermined time intervals for 4 or more months. In some instances, the PDE inhibitor is administered at predetermined time intervals for 5 or more months. In some instances, the PDE inhibitor is administered at predetermined time intervals for 6 or more months. In some instances, the PDE inhibitor is administered at predetermined time intervals for 7 or more months. In some instances, the PDE inhibitor is administered at predetermined time intervals for 8 or more months. In some instances, the PDE inhibitor is administered at predetermined time intervals for 9 or more months. In some instances, the PDE inhibitor is administered at predetermined time intervals for 10 or more months. In some instances, the PDE inhibitor is administered at predetermined time intervals for 11 or more months. In some instances, the PDE inhibitor is administered at predetermined time intervals for 12 or more months. In some instances, the PDE inhibitor is administered at predetermined time intervals for 24 or more months. In some instances, the PDE inhibitor is administered at predetermined time intervals for 36 or more months.

In some cases, a PDE inhibitor is administered intermittently for 1, 2, 3, 4, 5, 6, 7, 8, 9, 10, 14, 15, 28, 30 or more days. In some instances, the PDE inhibitor is administered intermittently for 1 or more days. In some instances, the PDE inhibitor is administered intermittently for 2 or more days. In some instances, the PDE inhibitor is administered intermittently for 3 or more days. In some instances, the PDE inhibitor is administered intermittently for 4 or more days. In some instances, the PDE inhibitor is administered intermittently for 5 or more days. In some instances, the PDE inhibitor is administered intermittently for 6 or more days. In some instances, the PDE inhibitor is administered intermittently for 7 or more days. In some instances, the PDE inhibitor is administered intermittently for 8 or more days. In some instances, the PDE inhibitor is administered intermittently for 9 or more days. In some instances, the PDE inhibitor is administered intermittently for 10 or more days. In some instances, the PDE inhibitor is administered intermittently for 14 or more days. In some instances, the PDE inhibitor is administered intermittently for 15 or more days. In some instances, the PDE inhibitor is administered intermittently for 28 or more days. In some instances, the PDE inhibitor is administered intermittently for 30 or more days.

In some embodiments, a PDE inhibitor is administered for at least 1 cycle, 2 cycles, 3 cycles, 4 cycles, 5 cycles, 6 cycles, 7 cycles, 8 cycles, or more. In some embodiments, a PDE inhibitor is administered for at least 1 cycle, 2 cycles, 3 cycles, 4 cycles, 5 cycles or more. In some cases, the PDE inhibitor is administered for at least 1 cycle. In some cases, the PDE inhibitor is administered for at least 2 cycles. In some cases, the PDE inhibitor is administered for at least 3 cycles. In some cases, the PDE inhibitor is administered for at least 4 cycles. In some cases, the PDE inhibitor is administered for at least 5 cycles. In some cases, the PDE inhibitor is administered for at least 6 cycles. In some cases, the PDE inhibitor is administered for at least 7 cycles. In some cases, the PDE inhibitor is administered for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, a PDE inhibitor is administered simultaneously or sequentially with an ICD inducer for at least 1 cycle, 2 cycles, 3 cycles, 4 cycles, 5 cycles, 6 cycles, 7 cycles, 8 cycles, or more. In some embodiments, a PDE inhibitor is administered simultaneously or sequentially with an ICD inducer for at least 1 cycle, 2 cycles, 3 cycles, 4 cycles, 5 cycles or more. In some cases, the PDE inhibitor is administered simultaneously or sequentially with an ICD inducer for at least 1 cycle. In some cases, the PDE inhibitor is administered simultaneously or sequentially with an ICD inducer for at least 2 cycles. In some cases, the PDE inhibitor is administered simultaneously or sequentially with an ICD inducer for at least 3 cycles. In some cases, the PDE inhibitor is administered simultaneously or sequentially with an ICD inducer for at least 4 cycles. In some cases, the PDE inhibitor is administered simultaneously or sequentially with an ICD inducer for at least 5 cycles. In some cases, the PDE inhibitor is administered simultaneously or sequentially with an ICD inducer for at least 6 cycles. In some cases, the PDE inhibitor is administered simultaneously or sequentially with an ICD inducer for at least 7 cycles. In some cases, the PDE inhibitor is administered simultaneously or sequentially with an ICD inducer for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, a PDE inhibitor is administered simultaneously or sequentially with an ICD inducer for at least 1, 5, 10, 14, 15, 20, 21, 28, 30, 60, or 90 days. In some cases, the PDE inhibitor is administered simultaneously or sequentially with an ICD inducer for at least 1 day. In some cases, the PDE inhibitor is administered simultaneously or sequentially with an ICD inducer for at least 5 days. In some cases, the PDE inhibitor is administered simultaneously or sequentially with an ICD inducer for at least 10 days. In some cases, the PDE inhibitor is administered simultaneously or sequentially with an ICD inducer for at least 14 days. In some cases, the PDE inhibitor is administered simultaneously or sequentially with an ICD inducer for at least 15 days. In some cases, the PDE inhibitor is administered simultaneously or sequentially with an ICD inducer for at least 20 days. In some cases, the PDE inhibitor is administered simultaneously or sequentially with an ICD inducer for at least 21 days. In some cases, the PDE inhibitor is administered simultaneously or sequentially with an ICD inducer for at least 28 days. In some cases, the PDE inhibitor is administered simultaneously or sequentially with an ICD inducer for at least 30 days. In some cases, the PDE inhibitor is administered simultaneously or sequentially with an ICD inducer for at least 60 days. In some cases, the PDE inhibitor is administered simultaneously or sequentially with an ICD inducer for at least 90 days.

In some instances, a PDE inhibitor is administered to a subject at a therapeutically effective amount. For example, the therapeutically effective amount is optionally administered in 1 dose, 2 doses, 3 doses, 4 doses, 5 doses, 6 doses or more. In some instances, the therapeutically effective amount of a PDE inhibitor is administered to a subject in 1 dose. In some instances, the therapeutically effective amount of a PDE inhibitor is administered to a subject in 2 or more doses. In some instances, the therapeutically effective amount of a PDE inhibitor is administered to a subject in 3 or more doses. In some instances, the therapeutically effective amount of a PDE inhibitor is administered to a subject in 4 or more doses. In some instances, the therapeutically effective amount of a PDE inhibitor is administered to a subject in 5 or more doses. In some instances, the therapeutically effective amount of a PDE inhibitor is administered to a subject in 6 or more doses.

In some cases, the therapeutically effective amount of the PDE inhibitor selectively inhibits hydrolysis of 2′3′-cGAMP.

In some embodiments, the therapeutically effective amount of the PDE inhibitor further reduces ATP hydrolysis in PDE by less than 50%, less than 40%, less than 30%, less than 20%, less than 10%, less than 5%, or by less than 1% relative to the ATP hydrolysis of a PDE in the absence of the PDE inhibitor. In some cases, the therapeutically effective amount of the PDE inhibitor reduces ATP hydrolysis in PDE by less than 50% relative to the ATP hydrolysis of a PDE in the absence of the PDE inhibitor. In some cases, the therapeutically effective amount of the PDE inhibitor reduces ATP hydrolysis in PDE by less than 40% relative to the ATP hydrolysis of a PDE in the absence of the PDE inhibitor. In some cases, the therapeutically effective amount of the PDE inhibitor reduces ATP hydrolysis in PDE by less than 30% relative to the ATP hydrolysis of a PDE in the absence of the PDE inhibitor. In some cases, the therapeutically effective amount of the PDE inhibitor reduces ATP hydrolysis in PDE by less than 20% relative to the ATP hydrolysis of a PDE in the absence of the PDE inhibitor. In some cases, the therapeutically effective amount of the PDE inhibitor reduces ATP hydrolysis in PDE by less than 10% relative to the ATP hydrolysis of a PDE in the absence of the PDE inhibitor. In some cases, the therapeutically effective amount of the PDE inhibitor reduces ATP hydrolysis in PDE by less than 5% relative to the ATP hydrolysis of a PDE in the absence of the PDE inhibitor. In some cases, the therapeutically effective amount of the PDE inhibitor reduces ATP hydrolysis in PDE by less than 4% relative to the ATP hydrolysis of a PDE in the absence of the PDE inhibitor. In some cases, the therapeutically effective amount of the PDE inhibitor reduces ATP hydrolysis in PDE by less than 3% relative to the ATP hydrolysis of a PDE in the absence of the PDE inhibitor. In some cases, the therapeutically effective amount of the PDE inhibitor reduces ATP hydrolysis in PDE by less than 2% relative to the ATP hydrolysis of a PDE in the absence of the PDE inhibitor. In some cases, the therapeutically effective amount of the PDE inhibitor reduces ATP hydrolysis in PDE by less than 1% relative to the ATP hydrolysis of a PDE in the absence of the PDE inhibitor. In some cases, the therapeutically effective amount of the PDE inhibitor does not induce ATP hydrolysis in PDE.

In some embodiments, the subject is a human.

In some embodiments, the subject is diagnosed with a cancer.

Method of Enhancing and/or Augmenting Type I IFN Production

In some embodiments, also described herein include a method of enhancing and/or augmenting type I interferon (IFN) production. In some instances, the method comprises an in vivo method. In some cases, the method differs from a systemic method because the production of IFNs is localized in the tumor microenvironment. In some cases, the method of enhancing type I interferon (IFN) production in a subject in need thereof, comprises administering to the subject a pharmaceutical composition comprising (i) an inhibitor of a 2′3′-cGAMP degradation polypeptide to block the hydrolysis of 2′3′-cGAMP; and (ii) a pharmaceutically acceptable excipient; wherein the presence of 2′3′-cGAMP activates the STING pathway, thereby enhancing the production of type I interferons.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, the cell has an elevated expression of PDE.

In some instances, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some embodiments, the inhibitor of a 2′3′-cGAMP degradation polypeptide is a PDE inhibitor. In some instances, the PDE inhibitor is a small molecule. In some instances, the PDE inhibitor is a PDE5 inhibitor. In some instances, the PDE inhibitor is a PDE10 inhibitor. In some instances, the PDE inhibitor is a Pan-PDE inhibitor. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

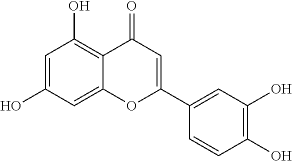 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

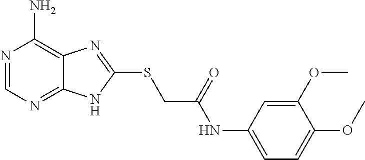 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

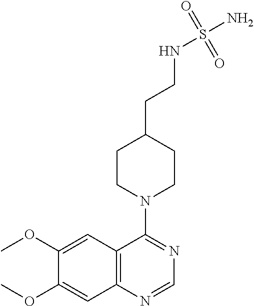 [SMILES: COc1cc2ncnc(N3CCC(CCNS(N)(=O)=O)CC3)c2cc1OC]

or a derivative, analog, or salt thereof.

In some embodiments, the subject has been administered an immunogenic cell death (ICD) inducer prior to administering the inhibitor of a 2′3′-cGAMP degradation polypeptide. In other instances, the subject is administered an immunogenic cell death (ICD) inducer after administering the inhibitor of a 2′3′-cGAMP degradation polypeptide or simultaneously with the inhibitor of a 2′3′-cGAMP degradation polypeptide.

In some cases, an ICD inducer comprises an agent that damages mitochondria resulting in the release of mtDNA. In some cases, an ICD inducer comprises an agent that induces micronuclei formation.

In some embodiments, an ICD inducer comprises radiation. In some cases, the radiation comprises UV radiation. In other cases, the radiation comprises γ radiation.

In some embodiments, an ICD inducer comprises a small molecule compound or a biologic. As described above, an ICD small molecule inducer optionally comprises a chemotherapeutic agent. In some cases, the chemotherapeutic agent comprises an anthracycline. In some cases, the anthracycline is doxorubicin or mitoxantrone. In some instances, the chemotherapeutic agent comprises a cyclophosphamide. In some instances, the cyclophosphamide is mafosfamide. In some embodiments, the chemotherapeutic agent is selected from bortezomib, daunorubicin, docetaxel, oxaliplatin, paclitaxel, or a combination thereof. In some cases, the ICD inducer comprises digitoxin or digoxin. In some cases, the ICD inducer comprises septacidin. In some cases, the ICD inducer comprises a combination of cisplatin and thapsigargin. In some cases, the ICD inducer comprises a combination of cisplatin and tunicamycin.

In some embodiments, an ICD inducer comprises a biologic (e.g., a protein-payload conjugate such as trastuzumab emtansine). In some cases, the ICD inducer comprises an activator of calreticulin (CRT) exposure.

In some embodiments, a PDE inhibitor is administered to a subject at least 0.5, 1, 1.5, 2, 3, 4, 5, 6, 7, 8, 9, 10, 11, 12, 18, 24, 36 or 48 hours after administration of the ICD inducer. In some cases, the PDE inhibitor is administered to the subject at least 0.5 hour after administration of the ICD inducer. In some cases, the PDE inhibitor is administered to the subject at least 1 hour after administration of the ICD inducer. In some cases, the PDE inhibitor is administered to the subject at least 1.5 hours after administration of the ICD inducer. In some cases, the PDE inhibitor is administered to the subject at least 2 hours after administration of the ICD inducer. In some cases, the PDE inhibitor is administered to the subject at least 3 hours after administration of the ICD inducer. In some cases, the PDE inhibitor is administered to the subject at least 4 hours after administration of the ICD inducer. In some cases, the PDE inhibitor is administered to the subject at least 5 hours after administration of the ICD inducer. In some cases, the PDE inhibitor is administered to the subject at least 6 hours after administration of the ICD inducer. In some cases, the PDE inhibitor is administered to the subject at least 7 hours after administration of the ICD inducer. In some cases, the PDE inhibitor is administered to the subject at least 8 hours after administration of the ICD inducer. In some cases, the PDE inhibitor is administered to the subject at least 9 hours after administration of the ICD inducer. In some cases, the PDE inhibitor is administered to the subject at least 10 hours after administration of the ICD inducer. In some cases, the PDE inhibitor is administered to the subject at least 11 hours after administration of the ICD inducer. In some cases, the PDE inhibitor is administered to the subject at least 12 hours after administration of the ICD inducer. In some cases, the PDE inhibitor is administered to the subject at least 18 hours after administration of the ICD inducer. In some cases, the PDE inhibitor is administered to the subject at least 24 hours after administration of the ICD inducer. In some cases, the PDE inhibitor is administered to the subject at least 36 hours after administration of the ICD inducer. In some cases, the PDE inhibitor is administered to the subject at least 48 hours after administration of the ICD inducer.

In some embodiments, a PDE inhibitor is administered to a subject at least 1, 2, 3, 4, 5, 6, 7, 8, 9, 10, 11, 12, 13, 14, 28, 30 or 40 days after administration of the ICD inducer. In some cases, the PDE inhibitor is administered to the subject at least 1 day after administration of the ICD inducer. In some cases, the PDE inhibitor is administered to the subject at least 2 days after administration of the ICD inducer. In some cases, the PDE inhibitor is administered to the subject at least 3 days after administration of the ICD inducer. In some cases, the PDE inhibitor is administered to the subject at least 4 days after administration of the ICD inducer. In some cases, the PDE inhibitor is administered to the subject 4 days after administration of the ICD inducer. In some cases, the PDE inhibitor is administered to the subject at least 5 days after administration of the ICD inducer. In some cases, the PDE inhibitor is administered to the subject at least 6 days after administration of the ICD inducer. In some cases, the PDE inhibitor is administered to the subject at least 7 days after administration of the ICD inducer. In some cases, the PDE inhibitor is administered to the subject at least 8 days after administration of the ICD inducer. In some cases, the PDE inhibitor is administered to the subject at least 9 days after administration of the ICD inducer. In some cases, the PDE inhibitor is administered to the subject at least 10 days after administration of the ICD inducer. In some cases, the PDE inhibitor is administered to the subject at least 11 days after administration of the ICD inducer. In some cases, the PDE inhibitor is administered to the subject at least 12 days after administration of the ICD inducer. In some cases, the PDE inhibitor is administered to the subject at least 13 days after administration of the ICD inducer. In some cases, the PDE inhibitor is administered to the subject at least 14 days after administration of the ICD inducer. In some cases, the PDE inhibitor is administered to the subject at least 28 days after administration of the ICD inducer. In some cases, the PDE inhibitor is administered to the subject at least 30 days after administration of the ICD inducer.

In some embodiments, a PDE inhibitor is administered to a subject at least 0.5, 1, 1.5, 2, 3, 4, 5, 6, 7, 8, 9, 10, 11, 12, 18, 24, 36 or 48 hours prior to administration of the ICD inducer. In some cases, the PDE inhibitor is administered to the subject at least 0.5 hour prior to administration of the ICD inducer. In some cases, the PDE inhibitor is administered to the subject at least 1 hour prior to administration of the ICD inducer. In some cases, the PDE inhibitor is administered to the subject at least 1.5 hours prior to administration of the ICD inducer. In some cases, the PDE inhibitor is administered to the subject at least 2 hours prior to administration of the ICD inducer. In some cases, the PDE inhibitor is administered to the subject at least 3 hours prior to administration of the ICD inducer. In some cases, the PDE inhibitor is administered to the subject at least 4 hours prior to administration of the ICD inducer. In some cases, the PDE inhibitor is administered to the subject at least 5 hours prior to administration of the ICD inducer. In some cases, the PDE inhibitor is administered to the subject at least 6 hours prior to administration of the ICD inducer. In some cases, the PDE inhibitor is administered to the subject at least 7 hours prior to administration of the ICD inducer. In some cases, the PDE inhibitor is administered to the subject at least 8 hours prior to administration of the ICD inducer. In some cases, the PDE inhibitor is administered to the subject at least 9 hours prior to administration of the ICD inducer. In some cases, the PDE inhibitor is administered to the subject at least 10 hours prior to administration of the ICD inducer. In some cases, the PDE inhibitor is administered to the subject at least 11 hours prior to administration of the ICD inducer. In some cases, the PDE inhibitor is administered to the subject at least 12 hours prior to administration of the ICD inducer. In some cases, the PDE inhibitor is administered to the subject at least 18 hours prior to administration of the ICD inducer. In some cases, the PDE inhibitor is administered to the subject at least 24 hours prior to administration of the ICD inducer. In some cases, the PDE inhibitor is administered to the subject at least 36 hours prior to administration of the ICD inducer. In some cases, the PDE inhibitor is administered to the subject at least 48 hours prior to administration of the ICD inducer.

In some embodiments, a PDE inhibitor is administered to a subject at least 1, 2, 3, 4, 5, 6, 7, 8, 9, 10, 11, 12, 13, 14, 28, 30 or 40 days prior to administration of the ICD inducer. In some cases, the PDE inhibitor is administered to the subject at least 1 day prior to administration of the ICD inducer. In some cases, the PDE inhibitor is administered to the subject at least 2 days prior to administration of the ICD inducer. In some cases, the PDE inhibitor is administered to the subject at least 3 days prior to administration of the ICD inducer. In some cases, the PDE inhibitor is administered to the subject at least 4 days prior to administration of the ICD inducer. In some cases, the PDE inhibitor is administered to the subject at least 5 days prior to administration of the ICD inducer. In some cases, the PDE inhibitor is administered to the subject at least 6 days prior to administration of the ICD inducer. In some cases, the PDE inhibitor is administered to the subject at least 7 days prior to administration of the ICD inducer. In some cases, the PDE inhibitor is administered to the subject at least 8 days prior to administration of the ICD inducer. In some cases, the PDE inhibitor is administered to the subject at least 9 days prior to administration of the ICD inducer. In some cases, the PDE inhibitor is administered to the subject at least 10 days prior to administration of the ICD inducer. In some cases, the PDE inhibitor is administered to the subject at least 11 days prior to administration of the ICD inducer. In some cases, the PDE inhibitor is administered to the subject at least 12 days prior to administration of the ICD inducer. In some cases, the PDE inhibitor is administered to the subject at least 13 days prior to administration of the ICD inducer. In some cases, the PDE inhibitor is administered to the subject at least 14 days prior to administration of the ICD inducer. In some cases, the PDE inhibitor is administered to the subject at least 28 days prior to administration of the ICD inducer. In some cases, the PDE inhibitor is administered to the subject at least 30 days prior to administration of the ICD inducer.

In some cases, a PDE inhibitor is administered simultaneously with an ICD inducer.

In some cases, a PDE inhibitor is administered continuously for 1, 2, 3, 4, 5, 6, 7, 8, 9, 10, 14, 15, 28, 30 or more days. In some instances, the PDE inhibitor is administered continuously for 1 or more days. In some instances, the PDE inhibitor is administered continuously for 2 or more days. In some instances, the PDE inhibitor is administered continuously for 3 or more days. In some instances, the PDE inhibitor is administered continuously for 4 or more days. In some instances, the PDE inhibitor is administered continuously for 5 or more days. In some instances, the PDE inhibitor is administered continuously for 6 or more days. In some instances, the PDE inhibitor is administered continuously for 7 or more days. In some instances, the PDE inhibitor is administered continuously for 8 or more days. In some instances, the PDE inhibitor is administered continuously for 9 or more days. In some instances, the PDE inhibitor is administered continuously for 10 or more days. In some instances, the PDE inhibitor is administered continuously for 14 or more days. In some instances, the PDE inhibitor is administered continuously for 15 or more days. In some instances, the PDE inhibitor is administered continuously for 28 or more days. In some instances, the PDE inhibitor is administered continuously for 30 or more days.

In some cases, a PDE inhibitor is administered at predetermined time intervals for 1, 2, 3, 4, 5, 6, 7, 8, 9, 10, 14, 15, 28, 30 or more days. In some instances, the PDE inhibitor is administered at predetermined time intervals for 1 or more days. In some instances, the PDE inhibitor is administered at predetermined time intervals for 2 or more days. In some instances, the PDE inhibitor is administered at predetermined time intervals for 3 or more days. In some instances, the PDE inhibitor is administered at predetermined time intervals for 4 or more days. In some instances, the PDE inhibitor is administered at predetermined time intervals for 5 or more days. In some instances, the PDE inhibitor is administered at predetermined time intervals for 6 or more days. In some instances, the PDE inhibitor is administered at predetermined time intervals for 7 or more days. In some instances, the PDE inhibitor is administered at predetermined time intervals for 8 or more days. In some instances, the PDE inhibitor is administered at predetermined time intervals for 9 or more days. In some instances, the PDE inhibitor is administered at predetermined time intervals for 10 or more days. In some instances, the PDE inhibitor is administered at predetermined time intervals for 14 or more days. In some instances, the PDE inhibitor is administered at predetermined time intervals for 15 or more days. In some instances, the PDE inhibitor is administered at predetermined time intervals for 28 or more days. In some instances, the PDE inhibitor is administered at predetermined time intervals for 30 or more days.

In some embodiments, a PDE inhibitor is administered at predetermined time intervals for 1, 2, 3, 4, 5, 6, 7, 8, 9, 10, 11, 12, 24, 36 or more months. In some instances, the PDE inhibitor is administered at predetermined time intervals for 1 or more month. In some instances, the PDE inhibitor is administered at predetermined time intervals for 2 or more months. In some instances, the PDE inhibitor is administered at predetermined time intervals for 3 or more months. In some instances, the PDE inhibitor is administered at predetermined time intervals for 4 or more months. In some instances, the PDE inhibitor is administered at predetermined time intervals for 5 or more months. In some instances, the PDE inhibitor is administered at predetermined time intervals for 6 or more months. In some instances, the PDE inhibitor is administered at predetermined time intervals for 7 or more months. In some instances, the PDE inhibitor is administered at predetermined time intervals for 8 or more months. In some instances, the PDE inhibitor is administered at predetermined time intervals for 9 or more months. In some instances, the PDE inhibitor is administered at predetermined time intervals for 10 or more months. In some instances, the PDE inhibitor is administered at predetermined time intervals for 11 or more months. In some instances, the PDE inhibitor is administered at predetermined time intervals for 12 or more months. In some instances, the PDE inhibitor is administered at predetermined time intervals for 24 or more months. In some instances, the PDE inhibitor is administered at predetermined time intervals for 36 or more months.

In some cases, a PDE inhibitor is administered intermittently for 1, 2, 3, 4, 5, 6, 7, 8, 9, 10, 14, 15, 28, 30 or more days. In some instances, the PDE inhibitor is administered intermittently for 1 or more days. In some instances, the PDE inhibitor is administered intermittently for 2 or more days. In some instances, the PDE inhibitor is administered intermittently for 3 or more days. In some instances, the PDE inhibitor is administered intermittently for 4 or more days. In some instances, the PDE inhibitor is administered intermittently for 5 or more days. In some instances, the PDE inhibitor is administered intermittently for 6 or more days. In some instances, the PDE inhibitor is administered intermittently for 7 or more days. In some instances, the PDE inhibitor is administered intermittently for 8 or more days. In some instances, the PDE inhibitor is administered intermittently for 9 or more days. In some instances, the PDE inhibitor is administered intermittently for 10 or more days. In some instances, the PDE inhibitor is administered intermittently for 14 or more days. In some instances, the PDE inhibitor is administered intermittently for 15 or more days. In some instances, the PDE inhibitor is administered intermittently for 28 or more days. In some instances, the PDE inhibitor is administered intermittently for 30 or more days.

In some embodiments, a PDE inhibitor is administered for at least 1 cycle, 2 cycles, 3 cycles, 4 cycles, 5 cycles, 6 cycles, 7 cycles, 8 cycles, or more. In some embodiments, a PDE inhibitor is administered for at least 1 cycle, 2 cycles, 3 cycles, 4 cycles, 5 cycles or more. In some cases, the PDE inhibitor is administered for at least 1 cycle. In some cases, the PDE inhibitor is administered for at least 2 cycles. In some cases, the PDE inhibitor is administered for at least 3 cycles. In some cases, the PDE inhibitor is administered for at least 4 cycles. In some cases, the PDE inhibitor is administered for at least 5 cycles. In some cases, the PDE inhibitor is administered for at least 6 cycles. In some cases, the PDE inhibitor is administered for at least 7 cycles. In some cases, the PDE inhibitor is administered for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, a PDE inhibitor is administered simultaneously or sequentially with an ICD inducer for at least 1 cycle, 2 cycles, 3 cycles, 4 cycles, 5 cycles, 6 cycles, 7 cycles, 8 cycles, or more. In some embodiments, a PDE inhibitor is administered simultaneously or sequentially with an ICD inducer for at least 1 cycle, 2 cycles, 3 cycles, 4 cycles, 5 cycles or more. In some cases, the PDE inhibitor is administered simultaneously or sequentially with an ICD inducer for at least 1 cycle. In some cases, the PDE inhibitor is administered simultaneously or sequentially with an ICD inducer for at least 2 cycles. In some cases, the PDE inhibitor is administered simultaneously or sequentially with an ICD inducer for at least 3 cycles. In some cases, the PDE inhibitor is administered simultaneously or sequentially with an ICD inducer for at least 4 cycles. In some cases, the PDE inhibitor is administered simultaneously or sequentially with an ICD inducer for at least 5 cycles. In some cases, the PDE inhibitor is administered simultaneously or sequentially with an ICD inducer for at least 6 cycles. In some cases, the PDE inhibitor is administered simultaneously or sequentially with an ICD inducer for at least 7 cycles. In some cases, the PDE inhibitor is administered simultaneously or sequentially with an ICD inducer for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, a PDE inhibitor is administered simultaneously or sequentially with an ICD inducer for at least 1, 5, 10, 14, 15, 20, 21, 28, 30, 60, or 90 days. In some cases, the PDE inhibitor is administered simultaneously or sequentially with an ICD inducer for at least 1 day. In some cases, the PDE inhibitor is administered simultaneously or sequentially with an ICD inducer for at least 5 days. In some cases, the PDE inhibitor is administered simultaneously or sequentially with an ICD inducer for at least 10 days. In some cases, the PDE inhibitor is administered simultaneously or sequentially with an ICD inducer for at least 14 days. In some cases, the PDE inhibitor is administered simultaneously or sequentially with an ICD inducer for at least 15 days. In some cases, the PDE inhibitor is administered simultaneously or sequentially with an ICD inducer for at least 20 days. In some cases, the PDE inhibitor is administered simultaneously or sequentially with an ICD inducer for at least 21 days. In some cases, the PDE inhibitor is administered simultaneously or sequentially with an ICD inducer for at least 28 days. In some cases, the PDE inhibitor is administered simultaneously or sequentially with an ICD inducer for at least 30 days. In some cases, the PDE inhibitor is administered simultaneously or sequentially with an ICD inducer for at least 60 days. In some cases, the PDE inhibitor is administered simultaneously or sequentially with an ICD inducer for at least 90 days.

In some instances, a PDE inhibitor is administered to a subject at a therapeutically effective amount. For example, the therapeutically effective amount is optionally administered in 1 dose, 2 doses, 3 doses, 4 doses, 5 doses, 6 doses or more. In some instances, the therapeutically effective amount of a PDE inhibitor is administered to a subject in 1 dose. In some instances, the therapeutically effective amount of a PDE inhibitor is administered to a subject in 2 or more doses. In some instances, the therapeutically effective amount of a PDE inhibitor is administered to a subject in 3 or more doses. In some instances, the therapeutically effective amount of a PDE inhibitor is administered to a subject in 4 or more doses. In some instances, the therapeutically effective amount of a PDE inhibitor is administered to a subject in 5 or more doses. In some instances, the therapeutically effective amount of a PDE inhibitor is administered to a subject in 6 or more doses.

In some cases, the therapeutically effective amount of the inhibitor of a 2′3′-cGAMP degradation polypeptide (e.g., a PDE inhibitor) selectively inhibits hydrolysis of 2′3′-cGAMP.

In some embodiments, the therapeutically effective amount of the inhibitor of a 2′3′-cGAMP degradation polypeptide (e.g., a PDE inhibitor) further reduces ATP hydrolysis in the 2′3′-cGAMP degradation polypeptide by less than 50%, less than 40%, less than 30%, less than 20%, less than 10%, less than 5%, or by less than 1% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 50% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 40% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 30% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 20% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 10% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 5% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 4% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 3% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 2% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) reduces ATP hydrolysis in 2′3′-cGAMP degradation polypeptide by less than 1% relative to the ATP hydrolysis of a 2′3′-cGAMP degradation polypeptide in the absence of the 2′3′-cGAMP degradation polypeptide inhibitor. In some cases, the therapeutically effective amount of the inhibitor of a 2′3′-cGAMP degradation polypeptide (e.g., a PDE inhibitor) does not induce ATP hydrolysis in 2′3′-cGAMP degradation polypeptide.

In some embodiments, a cancer described herein is a solid tumor. In some cases, exemplary solid tumors include breast cancer, lung cancer and glioblastoma (e.g., glioblastoma multiforme).

In some embodiments, a cancer described herein is a hematologic malignancy. In some instances, a hematologic malignancy is a leukemia, a lymphoma or a myeloma. In some cases, a hematologic malignancy is a B-cell malignancy.

In some embodiments, a cancer described herein is a relapsed or refractory cancer.

In some embodiments, a cancer described herein is a metastatic cancer.

Method of Inhibiting 2′3′-cGAMP Depletion

In some embodiments, further disclosed herein include methods of inhibiting depletion of 2′3′-cGAMP in a cell and selective inhibition of a 2′3′-cGAMP degradation polypeptide (e.g., ENPP-1). In some instances, a method of inhibiting depletion of 2′3′-cGAMP in a cell comprises contacting a cell comprising a 2′3′-cGAMP degradation polypeptide with an inhibitor to generate a 2′3′-cGAMP degradation polypeptide-inhibitor adduct, thereby inhibiting the 2′3′-cGAMP degradation polypeptide from degrading 2′3′-cGAMP to prevent the depletion of 2′3′-cGAMP in the cell.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, a method of selectively inhibits a phosphodiesterase (PDE) comprises contacting a cell characterized with an elevated population of cytosolic DNA with a PDE inhibitor to inhibit hydrolysis of 2′3-cGAMP, wherein the PDE inhibitor has a reduced inhibition function of ATP hydrolysis of the PDE. In some cases, the PDE inhibitor is a PDE5 inhibitor. In some cases, the PDE inhibitor is a PDE10 inhibitor. In some cases, the PDE inhibitor is a Pan-PDE inhibitor. In some cases, the PDE inhibitor is an ENPP-1 inhibitor. In some cases, the PDE inhibitor binds to the catalytic domain of ENPP-1. In some cases, the PDE inhibitor binds to the nuclease-like domain of ENPP-1.

In other instances, a method of selectively inhibits a phosphodiesterase (PDE) comprises contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE.

In additional instances, a method of selectively inhibits a phosphodiesterase (PDE) comprises contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE.

In some cases, the reduced inhibition function of ATP hydrolysis is relative to the ATP hydrolysis of a PDE in the absence of the PDE inhibitor. In some cases, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some instances, the PDE inhibitor reduces ATP hydrolysis in the PDE by less than 50% relative to the ATP hydrolysis of a PDE in the absence of the PDE inhibitor. In some instances, the PDE inhibitor reduces ATP hydrolysis in the PDE by less than 40% relative to the ATP hydrolysis of a PDE in the absence of the PDE inhibitor. In some instances, the PDE inhibitor reduces ATP hydrolysis in the PDE by less than 30% relative to the ATP hydrolysis of a PDE in the absence of the PDE inhibitor. In some instances, the PDE inhibitor reduces ATP hydrolysis in the PDE by less than 20% relative to the ATP hydrolysis of a PDE in the absence of the PDE inhibitor. In some instances, the PDE inhibitor reduces ATP hydrolysis in the PDE by less than 10% relative to the ATP hydrolysis of a PDE in the absence of the PDE inhibitor. In some instances, the PDE inhibitor reduces ATP hydrolysis in the PDE by less than 5% relative to the ATP hydrolysis of a PDE in the absence of the PDE inhibitor. In some instances, the PDE inhibitor reduces ATP hydrolysis in the PDE by less than 4% relative to the ATP hydrolysis of a PDE in the absence of the PDE inhibitor. In some instances, the PDE inhibitor reduces ATP hydrolysis in the PDE by less than 3% relative to the ATP hydrolysis of a PDE in the absence of the PDE inhibitor. In some instances, the PDE inhibitor reduces ATP hydrolysis in the PDE by less than 2% relative to the ATP hydrolysis of a PDE in the absence of the PDE inhibitor. In some instances, the PDE inhibitor reduces ATP hydrolysis in the PDE by less than 1% relative to the ATP hydrolysis of a PDE in the absence of the PDE inhibitor. In some cases, the PDE inhibitor does not inhibit ATP hydrolysis of the PDE.

In some embodiments, the cell has an elevated expression of PDE.

In some embodiments, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some instances, the PDE inhibitor is a small molecule. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

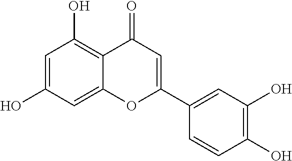 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

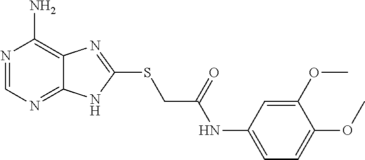 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

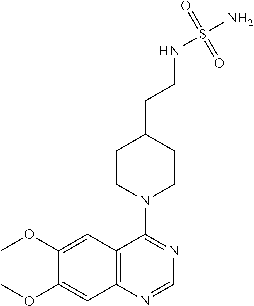 [SMILES: COc1cc2ncnc(N3CCC(CCNS(N)(=O)=O)CC3)c2cc1OC]

or a derivative, analog, or salt thereof.

In some instances, the cell comprises a cancer cell. In some instances, the cancer cell is a solid tumor cell (e.g., a breast cancer cell, a lung cancer cell or a cancer cell from glioblastoma). In other instances, the cancer cell is a cell from a hematologic malignancy (e.g., from a lymphoma, a leukemia, a myeloma or a B-cell malignancy).

In some embodiments, the cell comprises an effector cell. In some instances, the effector cell comprises a dendritic cell or a macrophage.

In some embodiments, the cell comprises a non-cancerous cell residing within a tumor microenvironment in which the cell comprises an elevated population of cytosolic DNA. In some cases, the cell comprises a non-cancerous cell residing within a tumor microenvironment in which the cGAS/STING pathway is activated.

In some embodiments, a subject is administered a recombinant vaccine comprising a vector that encodes a tumor antigen. In some instances, the subject administered a recombinant vaccine prior to administering the inhibitor of a 2′3′-cGAMP degradation polypeptide. In other instances, the subject is administered a recombinant vaccine after administering the inhibitor of a 2′3′-cGAMP degradation polypeptide or simultaneously with the inhibitor of a 2′3′-cGAMP degradation polypeptide.

In some embodiments, a nucleic acid vector described herein comprises a circular plasmid or a linear nucleic acid. In some cases, the circular plasmid or linear nucleic acid is capable of directing expression of a particular nucleotide sequence in an appropriate subject cell. In some cases, the vector has a promoter operably linked to the tumor antigen-encoding nucleotide sequence, which is operably linked to termination signals. In some instances, the vector also contains sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter, which can initiate transcription only when the host cell is exposed to some particular external stimulus.

In some instances, the vector is a plasmid. In some cases, the plasmid is useful for transfecting cells with nucleic acid encoding the tumor antigen, which the transformed host cells can be cultured and maintained under conditions wherein production of the tumor antigen takes place.

In some instances, the plasmid comprises a mammalian origin of replication in order to maintain the plasmid extrachromosomally and produce multiple copies of the plasmid in a cell. The plasmid can be pVAXI, pCEP4 or pREP4 from Invitrogen (San Diego, Calif.).

In some instances, the plasmid further comprises a regulatory sequence, which enables gene expression in a cell into which the plasmid is administered. In some cases, the coding sequence further comprises a codon that allows for more efficient transcription of the coding sequence in the host cell.

In some instances, the vector is a circular plasmid, which transforms a target cell by integration into the cellular genome or exist extrachromosomally (e.g., autonomous replicating plasmid with an origin of replication). Exemplary vectors include pVAX, pcDNA3.0, or provax, or any other expression vector capable of expressing DNA encoding the antigen and enabling a cell to translate the sequence to an antigen that is recognized by the immune system.

In some instances, the recombinant nucleic acid vaccine comprises a viral vector. Exemplary viral based vectors include adenoviral based, lentivirus based, adeno-associated (AAV) based, retroviral based, or poxvirus based vectors.

In some instances, the recombinant nucleic acid vaccine is a linear DNA vaccine, or linear expression cassette (“LEC”), that is capable of being efficiently delivered to a subject via electroporation and expressing one or more polypeptides disclosed herein. The LEC can be any linear DNA devoid of any phosphate backbone. The DNA can encode one or more microbial antigens. The LEC can contain a promoter, an intron, a stop codon, and/or a polyadenylation signal. In some cases, the LEC does not contain any antibiotic resistance genes and/or a phosphate backbone. In some cases, the LEC does not contain other nucleic acid sequences unrelated to the tumor antigen.

### Method of Activating a STING Protein Dimer

In some embodiments, a method of stabilizing a stimulator of interferon genes (STING) protein dimer in a cell comprises (a) contacting a cell characterized with an elevated expression of a phosphodiesterase (PDE) or an elevated population of cytosolic DNA with a PDE inhibitor to inhibit hydrolysis of 2′3′-cGAMP; and (b) interacting 2′3′-cGAMP to a STING protein dimer to generate a 2′3′-cGAMP-STING complex, thereby stabilizing the STING protein dimer. In some instances, interacting of 2′3′-cGAMP to a STING protein dimer to generate a 2′3′-cGAMP-STING complex further activates the STING protein dimer. In some cases, activation of the STING protein dimer further leads to upregulating the production of type I interferon (IFN). In some cases, the production of IFNs is localized in a tumor microenvironment.

In some instances, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, the PDE inhibitor is a small molecule. In some instances, the PDE inhibitor is a PDE5 inhibitor. In some instances, the PDE inhibitor is a PDE10 inhibitor. In some instances, the PDE inhibitor is a Pan-PDE inhibitor. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

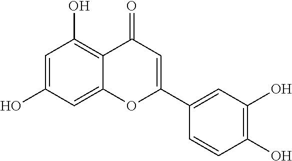 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

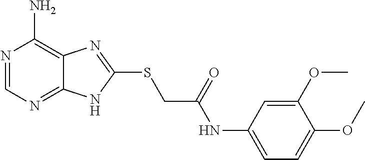 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

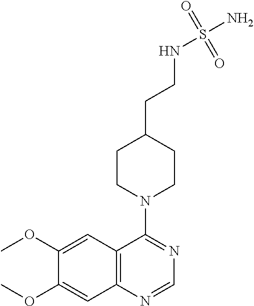 [SMILES: COc1cc2ncnc(N3CCC(CCNS(N)(=O)=O)CC3)c2cc1OC]

or a derivative, analog, or salt thereof.

In some instances, the cell comprises a cancer cell. In some instances, the cancer cell is a solid tumor cell (e.g., a breast cancer cell, a lung cancer cell or a cancer cell from glioblastoma). In other instances, the cancer cell is a cell from a hematologic malignancy (e.g., from a lymphoma, a leukemia, a myeloma or a B-cell malignancy).

In some embodiments, the cell comprises an effector cell. In some instances, the effector cell comprises a dendritic cell or a macrophage.

In some embodiments, the cell comprises a non-cancerous cell residing within a tumor microenvironment in which the cell comprises an elevated population of cytosolic DNA. In some cases, the cell comprises a non-cancerous cell residing within a tumor microenvironment in which the cGAS/STING pathway is activated.

### Additional Therapeutic Agents

In some embodiments, one or more methods described herein further comprising administering an additional therapeutic agent. In some instances, the additional therapeutic agent comprises a chemotherapeutic agent. In some instances, the additional therapeutic agent comprises an immune checkpoint inhibitor. Exemplary immune checkpoint inhibitor comprises an inhibitor of PD 1, an inhibitor of PD-L1, an inhibitor of TIM or an inhibitor of TIGIT. In some cases, the subject has a resistance to an immune checkpoint inhibitor prior to the administration of the inhibitor of PDE. In some cases, the PDE inhibitor and the additional therapeutic agent is administered simultaneously. In other cases, the PDE inhibitor and the additional therapeutic agent is administered sequentially. In some instances, the PDE inhibitor is administered before administering the additional therapeutic agent. In other instances, the PDE inhibitor is administered after administering the additional therapeutic agent.

### Pharmaceutical Compositions and Formulations

In certain embodiments, disclosed herein include pharmaceutical compositions and formulations comprising a compound described herein. In some embodiments, the pharmaceutical compositions described herein are formulated for administering to a subject by systemic administration. In other embodiments, the pharmaceutical compositions described herein are formulated for administering to a subject by local administration. In some instances, the administration routes include, but are not limited to, parenteral (e.g., intravenous, subcutaneous, intramuscular, intracerebral, intracerebroventricular, intra-articular, intraperitoneal, or intracranial), oral, sublingual, intranasal, buccal, rectal, or transdermal administration routes. In some instances, the pharmaceutical composition describe herein is formulated for parenteral (e.g., intravenous, subcutaneous, intramuscular, intracerebral, intracerebroventricular, intra-articular, intraperitoneal, or intracranial) administration. In other instances, the pharmaceutical composition describe herein is formulated for oral administration. In additional instances, the pharmaceutical composition describe herein is formulated for sublingual administration. In additional instances, the pharmaceutical composition describe herein is formulated for intranasal administration. In some cases, the pharmaceutical composition is administered to a subject as an injection. In other instances, the pharmaceutical composition is administered to a subject as an infusion.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens for a Pharmaceutical Composition

In some embodiments, a pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a PDE inhibitor, optionally with one or more additional therapeutic agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests. For example the term “treat” or “treating” with respect to tumor cells refers to stopping the progression of said cells, slowing down growth, inducing regression, or amelioration of symptoms associated with the presence of said cells.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

By “therapeutically effective amount” is meant an amount of a compound described herein effective to yield the desired therapeutic response. For example, an amount effective to delay the growth of or to cause a cancer, e.g., a lymphoma, or to shrink the cancer or prevent metastasis. The therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “derivative” refers to a chemically or biologically modified version of a chemical compound that is structurally similar to a parent compound and (actually or theoretically) derivable from that parent compound. In some cases, a derivative has different chemical or physical properties relative to the parent compound. For example, the derivative may be more hydrophilic or it may have altered reactivity as compared to the parent compound. Derivatization (i.e., modification) may involve substitution of one or more moieties within the molecule (e.g., a change in functional group) that do not substantially alter the function of the molecule for a desired purpose. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analogue” refers to a chemical compound that is structurally similar to another but differs slightly in composition (as in the replacement of one atom by an atom of a different element or in the presence of a particular functional group), but may or may not be derivable from the parent compound. A “derivative” differs from an “analogue” in that a parent compound may be the starting material to generate a “derivative,” whereas the parent compound may not necessarily be used as the starting material to generate an “analogue.”

### EXAMPLES

These examples are provided for illustrative purposes only and not to limit the scope of the claims provided herein.

### Example 1. ATP Hydrolysis

ENPP-1 is an ectonucleotidase which hydrolyze the STING substrate 2′,3′-cGAMP. In some instances, an inhibitor of ENPP-1 is capable of selectively blocking the hydrolysis of 2′,3′-cGAMP but reduces or minimally inhibits the hydrolysis of ATP. In some cases, an ATP hydrolysis assay is used to measure the selectivity of an ENPP-1 inhibitor. The following table 1 provides illustrative ENPP-1 inhibitors to be used with this experiment.

| ENPP-1 Inhibitor | Conc. 1 | Conc. 2 | Conc. 3 |
|---|---|---|---|
| ARL67156 | 10 μM | 100 μM | 1 mM |
| adenosine 5′-(α-borano)-β,γ-methylene | 10 μM | 100 μM | 1 mM |
| triphosphate | | | |
| N-(3,4-Dimethoxyphenyl)-2-(5-methoxy-3H- | 10 μM | 100 μM | 1 mM |
| imidazo[4,5-b]- pyridin-2-ylthio)acetamide | | | |

A 50 μL solution comprising 50 mM Tris-HCl, 200 mM NaCl, 0.1 mM CaCl₂, 1 ng/μL purified ENPP-1, and optionally with a ENPP-1 inhibitor, at pH 7.6, is prepared. The reaction is initiated with the addition of AMP-nP and is incubated for about 10 minutes at a temperature of about 37° C. The rate of product release is monitored continuously by measuring the OD at 405 nm. The specific activity is calculated as follows:

Specific activity (pmol/min/μg)=[Adjusted V max*(OD/min)*Conversion Factor # (pmo/OD)]/μg enzyme

# Conversion Factor is derived using calibration standard 4-Nitrophenol.

A control is prepared to establish background signal.

### Example 2. Indirect Quantitation of 2′,3′-cGAMP Hydrolysis

Hydrolysis of 2′,3′-cGMP generates GMP which subsequently releases free phosphate in the presence of a phosphatase. In some instances, the production of free phosphate is used to measure the selectivity of a ENPP-1 inhibitor.

A 50 μL solution comprising 50 mM Tris-HCl, 200 mM NaCl, 0.1 mM CaCl₂, 1 ng/μL purified ENPP-1, and optionally with a ENPP-1 inhibitor, at pH 7.6, is prepared. The reaction is initiated with the addition of 2′3′-cGAMP and is incubated for about 10 minutes at a temperature of about 37° C. The assay is stopped by adding a cocktail of MgCl₂, a chelator, an alkaline phosphatase and a ENPP-1 inhibitor. The rate of free phosphate is detected using a Malachite Green Phosphate Detection kit. The following table 2 provides illustrative ENPP-1 inhibitors to be used with this experiment.

| ENPP-1 Inhibitor | Conc. 1 | Conc. 2 | Conc. 3 |
|---|---|---|---|
| ARL67156 | 10 μM | 100 μM | 1 mM |
| adenosine 5′-(α-borano)-β,γ-methylene | 10 μM | 100 μM | 1 mM |
| triphosphate | | | |
| N-(3,4-Dimethoxyphenyl)-2-(5-methoxy-3H- | 10 μM | 100 μM | 1 mM |
| imidazo[4,5-b]-pyridin-2-ylthio)acetamide | | | |

The specific activity is calculated as follows:

Specific activity (pmol/min/μg)=[Adjusted V max*(OD/min)*Conversion Factor (pmo/OD)]/μg enzyme

### Example 3. In Silico Design of ENPP-1 Inhibitors

Ligand-based virtual screening is carried out with a known ENPP-1 inhibitor using the Schrödinger/E-pharmacophore modeling software. A 2D similarity search is conducted using Radial-ECFP-DL2 and MOLPRINT2D methods. An initial hit is set at 10,000 with subsequent refinements based on the number and strength of ligand-site residue interactions.

ENPP-1 PDB structures that are used during the in silico screening include 4GTW, 4GTX, 4GTY and 4GTZ.

### Example 4. Measuring ATP Hydrolysis by ENPP1

ENPP1 is an ectonucleotidase that hydrolyzes both the STING activator 2′,3′-cGAMP and 5′ATP (ATP). In some instances, an inhibitor of ENPP-1 is capable of selectively blocking the hydrolysis of 2′,3′-cGAMP while only minimally inhibiting the hydrolysis of ATP. The ATP analog p-Nitrophenyl 5′-Adenosine Monophosphate (AMP-pNP) has been demonstrated to accurately reflect the hydrolysis of the native substrate ATP by different classes of ENPP1 inhibitors, and was synthesized as described before (Lee at al. Substrate-Dependence of Competitive Nucleotide Pyrophosphatase/Phosphodiesterase 1 (NPP1) Inhibitors. Front Pharmacol. 2017 Feb. 15; 8:54). The ENPP1 assay with AMP-pNP substrate is conducted in a buffer containing 50 mM Tris-HCl (pH 8.5)/250 mM NaCl/0.5 mM CaCl₂/1 μM ZnCl₂/0.1% DMSO. Inhibitors are added at final concentrations ranging between 10 μM and 30 pM depending on the compound. Duplicate wells are run at each inhibitor concentration. The final assay volume is 40 μL and human recombinant ENPP1 is present at 60 ng/well. The assay is initiated by the addition of substrate (300 μM AMP-pNP final concentration), and incubated for 20 minutes at 37° C. The absorbance at 405 nm is then read in a Tecan® plate reader. Each assay plate also includes wells with no enzyme added (MIN OD) and wells with no inhibitor added (MAX OD). The percent inhibition of ENPP1 for each sample is then calculated as:

% inhibition={[Average of (MAX OD−MIN OD)−(sample OD−Average MIN OD)]/Average of (MAX OD−MIN OD)}×100%.

IC₅₀ values of compounds were calculated by entering the percent inhibition values into a sigmoidal variable slope nonlinear regression model in GraphPad Prism® software. IC50 values were converted to Ki values using the Cheng-Prusoff equation, where the Kₘ was 151 μM, based on internal determinations

$${K}_{i} = \frac{{IC}_{50}}{1 + \frac{[ S ]}{{K}_{m}}}$$

### Example 5. Quantitation of 2′,3′-cGAMP Hydrolysis

Hydrolysis of 2′,3′-cGAMP by ENPP1 generates the products 5′-GMP and 5′-AMP. In some instances, ENPP1 activity with 2′,3′-cGAMP substrate is measured using the AMP-Glo™ Assay kit (Promega Corporation) to quantitate the production of 5′-AMP. The AMP-Glo™ Assay Kit contains two reagents that are added sequentially. The first converts the 5′-AMP produced in the reaction to 5′ADP. The second converts the 5′-ADP to 5′ATP and reacts the 5′-ATP with the luciferase/luciferin pair to produce the luminescence signal. The ENPP1 assay with 2′,3′-cGAMP substrate is conducted in a buffer containing 50 mM Tris-HCl (pH 8.5)/250 mM NaCl/0.5 mM CaCl₂/1 μM ZnCl₂/0.1% DMSO. Inhibitors are added at final concentrations ranging between 10 μM and 30 pM depending on the compound. Duplicate wells are run at each inhibitor concentration. The final assay volume is 18 μL and human recombinant ENPP1 is present at 5 ng/well. The assay is initiated by the addition of substrate (20 μM 2′3′cGAMP final concentration), and incubated for 30 minutes at 37° C. To stop the reaction, 12 μl of AMP-Glo reagent I is added and the plate is incubated for 60 minutes at room temperature. 25 μl of AMP-detection reagent is then added and the wells are again incubated for 60 minutes at room temperature. The luminescence signal is then measured using a plate-reading luminometer. Each assay plate also includes wells with no enzyme added (MIN OD) and wells with no inhibitor added (MAX OD). The percent inhibition of ENPP1 for each sample is then calculated as:

% inhibition={[Average of (MAX OD−MIN OD)−(sample OD−Average MIN OD)]/Average of (MAX OD−MIN OD)}×100%.

IC50 values of compounds were calculated by entering the percent inhibition values into a sigmoidal variable slope nonlinear regression model in GraphPad Prism® software. IC50 values were converted to Ki values using the Cheng-Prusoff equation₂ where the Kₘ was 15 μM, based on internal determinations.

$${K}_{i} = \frac{{IC}_{50}}{1 + \frac{[ S ]}{{K}_{m}}}$$

### Example 6. PDE Inhibitors Library Screen on cGAMP Activated THP-1 Cells

Materials:

2′,3′-cGAMP (InvivoGen, catalog # tlrl-nacga23)—A STING agonist sensitive to hydrolysis by ENPP-1.

2′,3′-cGAM(PS)2 (Rp/Sp) (InvivoGen, cat # tlrl-nacga2srs)—A STING agonist resistant to hydrolysis by ENPP-1 (measure of maximum IFNβ response in absence of ENPP-1 degradation of STING agonist).

IFNβ Assay Kit: VeriKine Human Interferon Beta ELISA Kit (PBL Assay Science, catalog #41410). Standard range in the kit (pg/mL): 50, 100, 200, 400, 1000, 2000, 4000.

Controls:

Negative control: unstimulated THP-1 cells (no 2′,3′-cGAMP or 2′,3′-cGAMP(PS)2(Rp/Sp)).

Vehicle control: 0.1% DMSO (control where no compounds were added—vehicle used to dissolve compounds. 10 uL media (control used for wells where 2′,3′-cGAMP or 2′,3′-cGAMP(PS)2(Rp/Sp) was not added))

Positive controls: 2′,3′-cGAMP sensitive to ENPP1 hydrolysis and 2′,3′-cGAM(PS)2 (Rp/Sp) insensitive to ENPP1 hydrolysis

IFNβ Sample Analysis Dilution Factor:

Neat samples (no dilution) analyzed for IFNβ in samples of 50 μL.

THP-1 Cell Activation and Screening of Test Compounds:
(a) THP-1 cells from bulk cultures were counted and suspended in RPMI 1640, 20% FBS, 2.5 mM L-alanyl-L-glutamine at a concentration of 5.5×10⁶ cells/mL. The THP-1 cells were subsequently seeded into 96 well round bottom plates—volume of 180 μL/well (1×10⁶ cells per well), and the plate was incubated for 1 hour at 37° C., 5% CO₂.
(b) Test compounds comprising known phosphodiesterase inhibitors (PDEs), were screened in triplicate (N1, N2, N3) on THP-1 cells. For compound screening, test compounds were assayed at a final concentration of 10 μM. A 200 μM (0.2 mM) working solution of each compound was prepared by diluting 10 mM DMSO stock solutions of each compound in media at a dilution of 50:1 (1640, 20% FBS, 2.5 mM L-alanyl-L-glutamine). Working solutions of each test compound were added to triplicate wells containing 180 uL of THP-1 cell suspension, in a volume of 10 μL to achieve a 10 μM concentration following the addition of a 10 μL volume 2′,3′-cGAMP in the final step to activate the STING pathway.
(c) The STING agonist 2′,3′-cGAMP ligand provided as a sterile powder by InvivoGen was diluted in sterile, endotoxin-free water, as per the manufacturer's instructions to yield a 1 mg/mL solution (1.4 mM or 1400 μM). Working solutions of 2′,3′-cGAMP at concentrations of 600 and 800 or 1000 μM (20× the final concentrations used to activate the STING pathway in the THP-1 cells) were prepared by making the small dilutions of the 1 mg/mL stock of 2′,3′-cGAMP with the cell culture media.
(d) Following a 1-hour pre-incubation of the cells with the various test compounds, the STING pathway was activated by the addition of 2′,3′-cGAMP. Final concentrations of 2′,3′-cGAMP used to activate the THP-1 cells were 30 μM and a higher concentration of 40 or 50 μM. Vehicle control wells where 2′,3′-cGAMP ligand is added at concentrations of 30 and 50 μM in the absence of any test compounds but 0.1% DMSO were included to assess baseline activation of the THP-1 cells.
(e) The nonhydrolyzable form of 2′,3′-cGAMP or 2′,3′-cGAM(PS)2 (Rp/Sp) was included as an additional positive control-tested at concentrations of 40, and 80 μM in duplicate or triplicate. This agonist represents maximum activation of the STING pathway in the absence of the agonist degradation as occurs with native 2′,3′-cGAMP, with an IFNβ response higher than the response seen with native 2′,3′-cGAMP.
(f) “Vehicle only” control wells in triplicate were included on each plate to assess the basal level of STING activation in the absence of agonist.
(g) Final assay volume were 200 μL/well
   1) THP-1 cells=180 μL
   2) test compounds=10 μL
   3) 2′,3′-cGAMP or 2′,3′-cGAM(PS)2 (Rp/Sp) or vehicle control=10 μL
(h) Plates were incubated for 24 hours at 37° C., 5% CO₂.
(i) Cell culture supernatants were harvested by centrifuging the plates at 200×g for 10 minutes. Cell culture supernatants were then transferred to a clean plate and stored at −80° C. until ready to analyze for IFNβ levels.
(j) IFNβ levels in cell culture supernatants were determined by ELISA according to the manufacturer's instructions (VeriKine Human IFNβ Assay).
(k) The interpolated data was normalized to vehicle control, or unstimulated control, and analyzed.

Results:

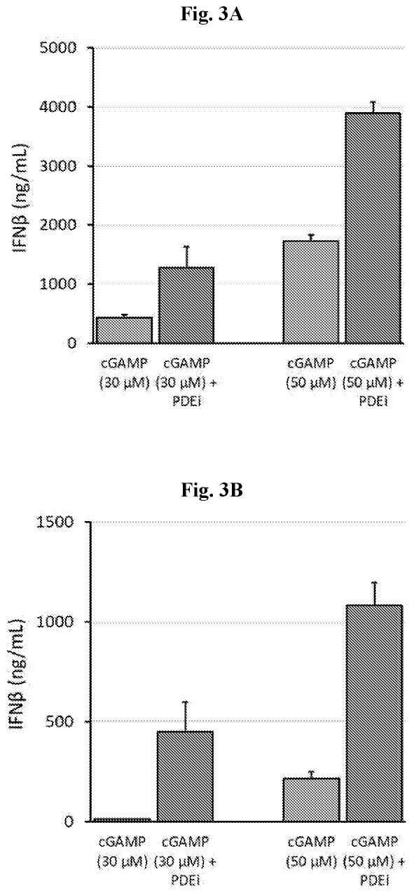

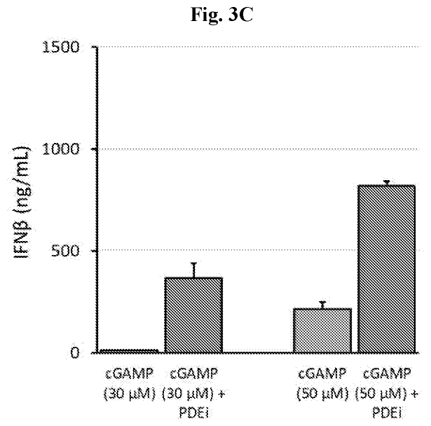

FIG. 3A-FIG. 3C illustrate exemplary compounds identified in the screen that augment cGAMP mediated IFNβ production.

Compounds found to augment IFNβ production in THP-1 cells activated with a suboptimal concentration of 2′,3′-cGAMP were evaluated for inhibition of ENPP1 mediated hydrolysis of 2′3′-cGAMP, as described in Example 5 using the AMP-GLO method. The following table 3 exemplifies the compounds that are inhibitors of ENPP1. Inhibition of 2′3′-cGAMP hydrolysis by ENPP1 in the presence of Compounds 1-3 at concentrations of 1 and 10 μM is shown.

| | Inhibition of ENPP1 with |
|---|---|
| Compound | cGAMP as the Substrate |
| 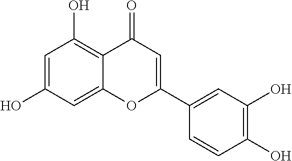 [SMILES: O=c1cc(-c2ccc(O)c(O)c2)oc2cc(O)cc(O)c12] Compound 1 | 99% Inhibition at 10 μM 76% Inhibition at 1 μM |
| 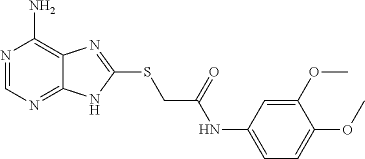 [SMILES: COc1ccc(NC(=O)CSc2nc3c(N)ncnc3[nH]2)cc1OC] Compound 2 | 99% Inhibition at 10 μM 99% Inhibition at 1 μM |
| 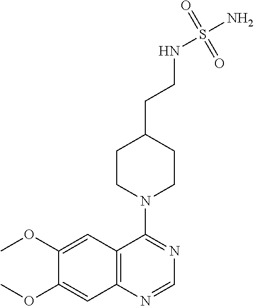 [SMILES: COc1cc2ncnc(N3CCC(CCNS(N)(=O)=O)CC3)c2cc1OC] Compound 3 | 99% Inhibition at 10 μM 99% Inhibition at 1 μM |

Inhibitor Selectivity

ENPP-1 catalyzes the hydrolysis of both 2′3′-cGAMP and ATP substrates. Compounds were tested for inhibition of ENPP-1 mediated hydrolysis of both the 2′3′-cGAMP and AMP-pNP (an analog of ATP) substrate to assess compound selectivity using methods described in Examples 4 and 5. In table 4 below, the potency of compounds to inhibit 2′3′cGAMP and AMP-pNP substrate hydrolysis by ENPP1 is provided as Ki determinations (nM). Additionally, the selectivity ratio for 2′3′c-GAMP versus AMP-pNP substrate inhibition has been calculated [Ki (AMP-pNP)/Ki (2′3′-cGAMP)]. The selectivity for inhibition of cGAMP over AMP-pNP hydrolysis by ENPP1 ranges from ˜6.8-fold for Compound 25, up to >37,500-fold for Compound 4. These results demonstrate that it is possible to identify inhibitors of ENPP-1 that selectively block the hydrolysis of 2′3′-cGAMP, while having a limited effect on the hydrolysis of the ATP analog.

| | | | | | Substrate |
|---|---|---|---|---|---|
| | | | | | Selectivity |
| | | | ENPP1 inhibition | | Ki Ratio |
| | | | Ki (nM) | | AMP- |
| | | | cGAMP | AMP-pNP | pNP/2′3′cGA |
| Compound | Structure | Series | Substrate | Substrate | MP |
| Compound 2 | 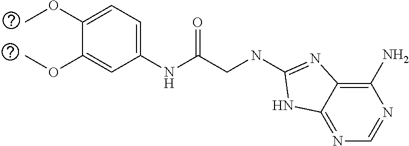 [SMILES: COc1ccc(NC(=O)CNc2nc3c(N)ncnc3[nH]2)cc1OC] | 2 | 1.2 | >3000 | >2500 |
| Compound 3 | 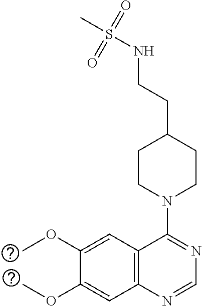 [SMILES: COc1cc2ncnc(N3CCC(CCNS(C)(=O)=O)CC3)c2cc1OC] | 1 | 4.4 | 155 | 35.2 |
| Compound 4 | 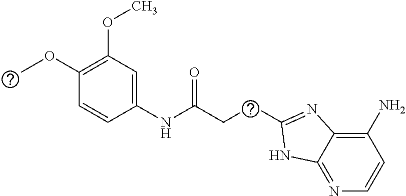 [SMILES: COc1ccc(NC(=O)CCc2nc3c(N)ccnc3[nH]2)cc1OC] | 2 | 0.8 | >30000 | >37500 |
| Compound 5 | 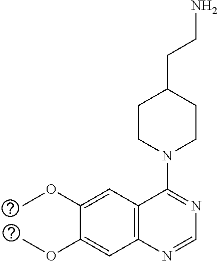 [SMILES: COc1cc2ncnc(N3CCC(CCN)CC3)c2cc1OC] | 1 | 46.7 | >30000 | >642 |
| Compound 6 | 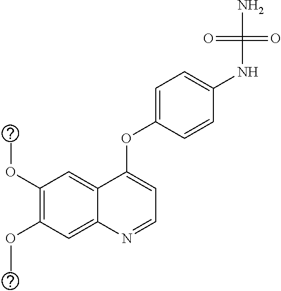 | 1 | 30.7 | >10000 | >325 |
| Compound 7 | 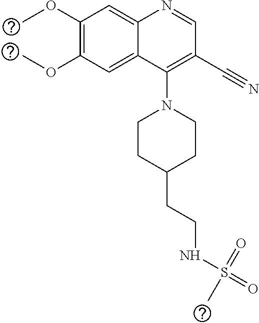 [SMILES: COc1cc2ncc(C#N)c(N3CCC(CCNS(C)(=O)=O)CC3)c2cc1OC] | 1 | 0.7 | 171 | 244 |
| Compound 8 | 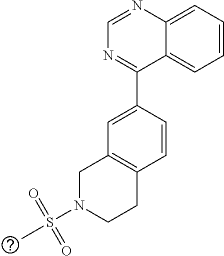 [SMILES: CS(=O)(=O)N1CCc2ccc(-c3ncnc4ccccc34)cc2C1] | 1 | 190 | >30000 | >158 |
| Compound 9 | 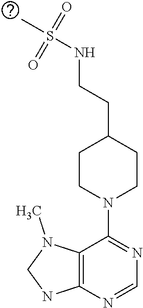 [SMILES: CN1CNc2ncnc(N3CCC(CCNS(C)(=O)=O)CC3)c21] | 1 | 77.7 | >10000 | >128 |
| Compound 10 | 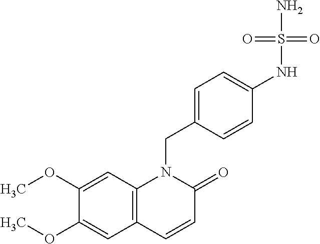 [SMILES: COc1cc2ccc(=O)n(Cc3ccc(NS(N)(=O)=O)cc3)c2cc1OC] | 1 | 234 | >30000 | >127 |
| Compound 11 | 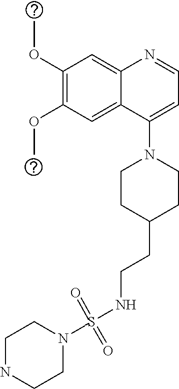 | 1 | 257 | >30000 | >116 |
| Compound 12 | 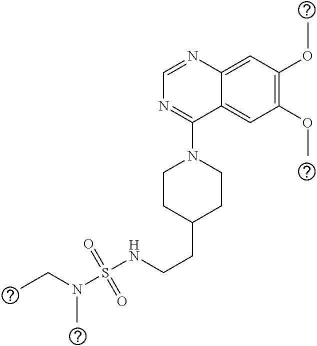 [SMILES: CCN(C)S(=O)(=O)NCCC1CCN(c2ncnc3cc(OC)c(OC)cc23)CC1] | 1 | 336 | >30000 | >89.0 |
| Compound 13 | 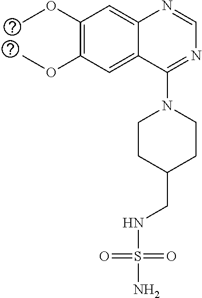 [SMILES: COc1cc2ncnc(N3CCC(CNS(N)(=O)=O)CC3)c2cc1OC] | 1 | 2.4 | 208 | 86.7 |
| Compound 14 | 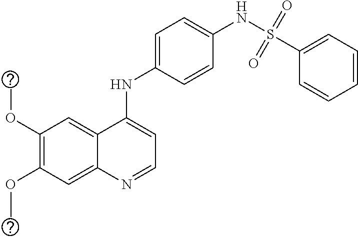 [SMILES: COc1cc2nccc(Nc3ccc(NS(=O)(=O)c4ccccc4)cc3)c2cc1OC] | 1 | 29.1 | 1830 | 62.9 |
| Compound 15 | 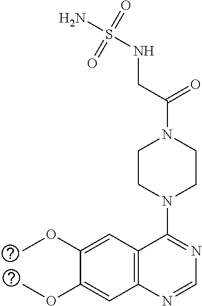 [SMILES: COc1cc2ncnc(N3CCN(C(=O)CNS(N)(=O)=O)CC3)c2cc1OC] | 1 | 7.3 | 418 | 57.3 |
| Compound 16 | 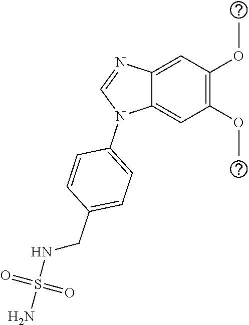 [SMILES: COc1cc2ncn(-c3ccc(CNS(N)(=O)=O)cc3)c2cc1OC] | 1 | 27.1 | 1460 | 53.9 |
| Compound 17 | 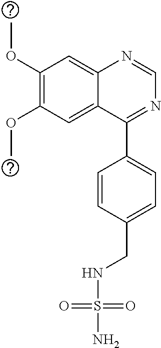 [SMILES: COc1cc2ncnc(-c3ccc(CNS(N)(=O)=O)cc3)c2cc1OC] | 1 | 2.9 | 140 | 48.3 |
| Compound 18 | 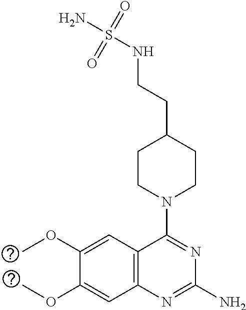 [SMILES: COc1cc2nc(N)nc(N3CCC(CCNS(N)(=O)=O)CC3)c2cc1OC] | 1 | 6.2 | 296 | 47.7 |
| Compound 19 | 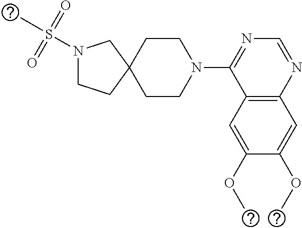 [SMILES: COc1cc2ncnc(N3CCC4(CC3)CCN(S(C)(=O)=O)C4)c2cc1OC] | 1 | 0.8 | 35.8 | 44.8 |
| Compound 20 | 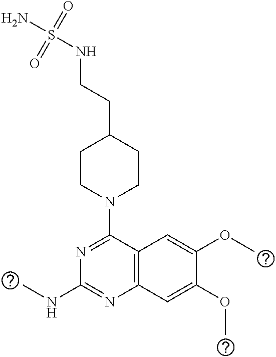 [SMILES: CNc1nc(N2CCC(CCNS(N)(=O)=O)CC2)c2cc(OC)c(OC)cc2n1] | 1 | 77.5 | >3000 | >38.7 |
| Compound 21 | 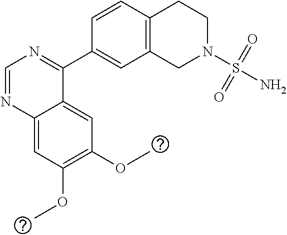 [SMILES: COc1cc2ncnc(-c3ccc4c(c3)CN(S(N)(=O)=O)CC4)c2cc1OC] | 1 | 1.8 | 65.2 | 36.2 |
| Compound 22 | 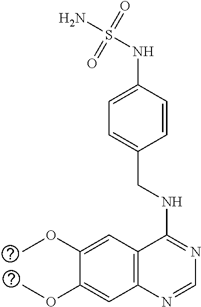 [SMILES: COc1cc2ncnc(NCc3ccc(NS(N)(=O)=O)cc3)c2cc1OC] | 1 | 9.1 | 316 | 34.7 |
| Compound 23 | 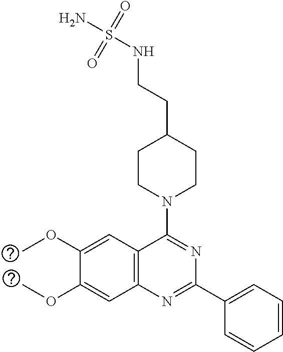 [SMILES: COc1cc2nc(-c3ccccc3)nc(N3CCC(CCNS(N)(=O)=O)CC3)c2cc1OC] | 1 | 418 | >10000 | >23.9 |
| Compound 24 | 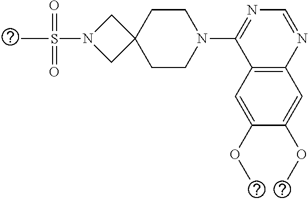 [SMILES: COc1cc2ncnc(N3CCC4(CC3)CN(S(C)(=O)=O)C4)c2cc1OC] | 1 | 60.5 | 1130 | 18.7 |
| Compound 25 | 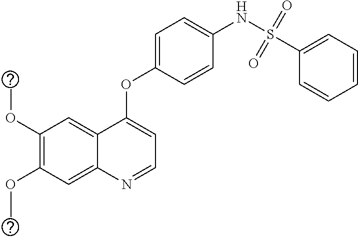 [SMILES: COc1cc2nccc(Oc3ccc(NS(=O)(=O)c4ccccc4)cc3)c2cc1OC] | 1 | 44 | >300 | >6.80 |

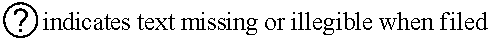

cGAMP Substrate:

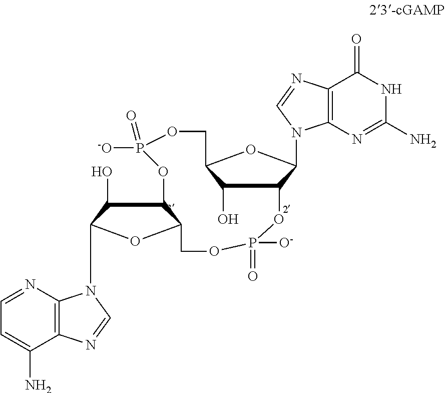

Amp-pNP Substrate:

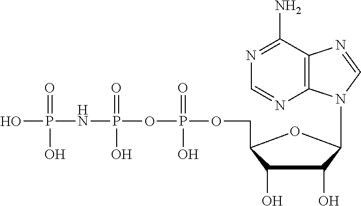

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a subject having a cancer primed by an immunogenic cell death (ICD) inducer, comprising:
administering to the subject a phosphodiesterase (PDE) inhibitor, wherein the PDE inhibitor prevents hydrolysis of 2′3′-cGAMP.

2. The method of claim 1, wherein the PDE comprises an ectonucleotide pyrophosphatase/phosphodiesterase (ENPP) protein.

3. The method of claim 2, wherein the ENPP protein comprises ectonucleotide pyrophosphatase/phosphodiesterase family member 1 (ENPP-1).

4. The method of claim 1, wherein the PDE inhibitor is a small molecule.

5. The method of claim 1, wherein the PDE inhibitor is a ENPP-1 inhibitor.

6. The method of claim 1, wherein the PDE inhibitor is a reversible inhibitor, a competitive inhibitor, an allosteric inhibitor, a mixed inhibitor, or an irreversible inhibitor.

7. The method of claim 1, wherein the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative, PSB-POM1412-(3H-imidazo[4,5-b]pyridin-2-ylthio)-N-(3,4-dimethoxyphenyl)acetamide or a derivative, analog, or salt thereof; 2-(6-Amino-9H-purin-8-ylthio)-N-(3,4-dimethoxyphenyl)-acetamide, or a salt thereof; N-(3,4-Dimethoxyphenyl)-2-(5-methoxy-3H-imidazo[4,5-b]-pyridin-2-ylthio)acetamide or a salt thereof; 2-(1-(6,7-Dimethoxyquinazolin-4-yl)piperidin-4-yl)ethyl sulfamide or a salt thereof; ((1-(6,7-Dimethoxyquinazolin-4-yl)piperidin-4-yl)methyl)sulfamide or a salt thereof; or SK4A (SAT0037) or a derivative or salt thereof.

8. The method of claim 1, wherein the PDE inhibitor comprises Compound 1, Compound 2, Compound 3, or a derivative, analog, or salt thereof.

9. The method of claim 1, wherein the cancer is a solid tumor comprising breast cancer, lung cancer or glioblastoma, or a hematologic malignancy.

10. The method of claim 1, wherein the cancer is a hematologic malignancy comprising a leukemia, a lymphoma or a myeloma.

11. The method of claim 1, wherein the immunogenic cell death (ICD) inducer comprises radiation, a small molecule compound or a biologic, or a chemotherapeutic agent.

12. The method of claim 1, wherein the PDE inhibitor is administered continuously, at predetermined time intervals or intermittently for 1, 2, 3, 4, 5, 6, 7, 8, 9, 10, 14, 15, 28, 30 or more days.

13. The method of claim 1, wherein the PDE inhibitor is administered to the subject at a therapeutically effective amount.

14. The method of claim 13, wherein the therapeutically effective amount of the PDE inhibitor selectively inhibits hydrolysis of 2′3′-cGAMP.

15. The method of claim 13, wherein the therapeutically effective amount of the PDE inhibitor reduces ATP hydrolysis in PDE by less than 50%, less than 40%, less than 30%, less than 20%, or by less than 10% relative to the ATP hydrolysis of a PDE in the absence of the PDE inhibitor.

16. A method of enhancing type I interferon (IFN) production in a subject in need thereof, comprising:
administering to the subject a pharmaceutical composition comprising:
i) an inhibitor of a 2′3′-cGAMP degradation polypeptide to block the hydrolysis of 2′3′-cGAMP; and
ii) a pharmaceutically acceptable excipient;
wherein the presence of 2′3′-cGAMP activates the STING pathway, thereby enhancing the production of type I interferons.

17. The method of claim 16, wherein the production of IFNs is localized in a tumor microenvironment.

18. The method of claim 16, wherein the 2′3′-cGAMP degradation polypeptide is a phosphodiesterase (PDE).

19. The method of claim 16, wherein the 2′3′-cGAMP degradation polypeptide is an ectonucleotide pyrophosphatase/phosphodiesterase (ENPP) protein.

20. The method of claim 16, wherein the 2′3′-cGAMP degradation polypeptide is ectonucleotide pyrophosphatase/phosphodiesterase family member 1 (ENPP-1).

21. The method of claim 16, wherein the cell has an elevated expression of PDE.

22. The method of claim 16, wherein the cell has an elevated population of cytosolic DNA generated by an ICD-mediated event.

23. The method of claim 16, wherein the inhibitor is a PDE inhibitor.

24. The method of claim 23, wherein the PDE inhibitor is a small molecule.

25. The method of any one of claims 23 or 24, wherein the PDE inhibitor is an ENPP-1 inhibitor.

26. The method of any one of the claims 16 or 23-25, wherein the PDE inhibitor is a reversible inhibitor, a competitive inhibitor, an allosteric inhibitor, a mixed inhibitor, or an irreversible inhibitor.

27. The method of any one of the claims 16 or 23-26, wherein the PDE inhibitor comprises comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative, PSB-POM1412-(3H-imidazo[4,5-b]pyridin-2-ylthio)-N-(3,4-dimethoxyphenyl)acetamide or a derivative, analog, or salt thereof; 2-(6-Amino-9H-purin-8-ylthio)-N-(3,4-dimethoxyphenyl)-acetamide, or a salt thereof; N-(3,4-Dimethoxyphenyl)-2-(5-methoxy-3H-imidazo[4,5-b]-pyridin-2-ylthio)acetamide or a salt thereof; 2-(1-(6,7-Dimethoxyquinazolin-4-yl)piperidin-4-yl)ethyl sulfamide or a salt thereof; ((1-(6,7-Dimethoxyquinazolin-4-yl)piperidin-4-yl)methyl)sulfamide or a salt thereof; or SK4A (SAT0037) or a derivative or salt thereof.

28. The method of one of the claims 16 or 23-26, wherein the PDE inhibitor comprises Compound 1, Compound 2, Compound 3, or a derivative, analog, or salt thereof.

29. The method of claim 16, wherein the subject has been administered an immunogenic cell death (ICD) inducer prior to or simultaneously with the inhibitor of a 2′3′-cGAMP degradation polypeptide.

30. The method of any one of the claims 16-29, wherein the inhibitor of a 2′3′-cGAMP degradation polypeptide is administered to the subject at a therapeutically effective amount.

31. The method of claim 30, wherein the therapeutically effective amount of the inhibitor of a 2′3′-cGAMP degradation polypeptide selectively inhibits hydrolysis of 2′3′-cGAMP but not ATP hydrolysis in the 2′3′-cGAMP degradation polypeptide.

32. The method of any one of the claims 16-31, wherein the subject is diagnosed with cancer.
